(19) United States
(12) Patent Application Publication
(10) Pub. No.: US 2024/0269088 A1
(43) Pub. Date: Aug. 15, 2024

(54) PAIN RELIEF PATCH

(71) Applicant: CS MEDICA A/S, Copenhagen (DK)

(72) Inventor: Lone HENRIKSEN, Fredensborg (DK)

(21) Appl. No.: 18/564,563

(22) PCT Filed: May 25, 2022
(86) PCT No.: PCT/EP2022/064268

§ 371 Date: Nov. 27, 2023

(30) Foreign Application Priority Data
May 25, 2021 (DK) PA202170269

(51) Int. Cl. A61K 9/70; A61K 31/00; A61K 31/045; A61K 36/28; A61P 29/00

(52) U.S. Cl. CPC A61K 9/7046; A61K 31/045; A61K 31/658; A61K 36/28; A61P 29/00

(57) ABSTRACT

The present invention concerns a skin patch, such as a dermal and/or transdermal pain relief patch comprising a backing layer (10), an adhesive layer (20), and optionally a release liner (30), said adhesive layer (20) comprising: one or more cannabinoid(s) present in an amount of 0.1-20%. Arnica extract and/or camphor in an amount of 0.2-6%, menthol in an amount of 0.2-6%, such as 0.5-5%, or 1.0-3% (by weight); and acrylates copolymer in an amount of at least 60, 70, 80, or 90% (by weight). Furthermore, a production method for such a skin patch, and different uses and/or application of such skin patches are disclosed.

### FIELD OF THE INVENTION

The present invention relates to a medical device in the form of a skin patch, such as a dermal and/or transdermal patch for use in the treatment and/or alleviation of pain and aches. Pain treatment comprises application of a patch comprising a cannabinoid-comprising composition onto the skin of an effected area in a subject, such as a sore muscle, joint, tendon, cartilage, or the like. In some embodiments, the cannabinoid is selected from one or more of cannabidiol (CBD), cannabigerol (CBG), and/or cannabinol (CBN). Further active components may comprise one or more of Arnica extract, Camphor, and/or Menthol.

### BACKGROUND OF THE INVENTION

Cannabinoids such as cannabidiol (CBD; CAS no. 3956-29-1) have been associated with wound healing, pain relief and anti-inflammatory effects.

There is a need for compositions and formulations for efficient pain management.

WO2020024056 discloses compositions comprising cannabinoids and absorbable material and uses thereof.

WO2015161165 concerns a transdermal cannabinoid patch.

US20200138737 relates to compositions comprising cannabinoids for relief of pain.

WO2020136593 pertains to herbal preparation-enriched cannabinoid compositions and methods of treatment.

US20200282062 concerns devices for administering CBD or CBG and a plant oil for treatment of seizure, insomnia, or anxiety.

US 202034657 related to transdermal cannabinoid formulations including a versatile cannabinoid stock provided as a variety of cosmetic delivery systems providing enhanced cannabinoid absorption, and more controlled release into vascular and/or lymphatic systems for greater efficacy and a desirable subject experience.

Pain medication is generally not 100% effective, and numbers ranging from 20-70% have been reported. Common pain treatment comprises enteral administration of an analgesic, usually by oral intake of pain killers, such as paracetamol, or NSAIDs (nonsteroidal anti-inflammatory drugs, such as aspirin, ibuprofen or diclofenac).

There is a need for more specific, non-systemic routes of administration, such as via topical administration of active ingredients through dermal patches as disclosed herein.

### SUMMARY OF THE INVENTION

As presented herein, surprisingly and/or unexpectedly, and from a wide range of component candidates and concentration ranges, the inventor has found the following compositions to be effective in relation to pain management and/or pain treatment, and compatible with administration by a skin patch, e.g. formulated as a dermal- and/or transdermal patch

In a first aspect, the present invention concerns a dermal and/or transdermal patch comprising, cannabinoid(s). Such a patch can e.g. be formulated as a pain relief patch, said patch comprising a backing layer, an adhesive layer, and optionally a release liner (30), said adhesive layer comprising (by weight):
Acrylates copolymer in an amount of at least 60, 70, 80, or 90%;
One or more cannabinoid(s) such as cannabidiol (CBD), cannabigerol (CBG), and/or cannabinol (CBN) present in an amount of 0.1-20%, such as 0.1-10%, or 0.2-5%;
Arnica extract and/or camphor in an amount of 0.2-6%, such as 0.5-5%, or 1.0-3%; and
Menthol in an amount of 0.2-6%, such as 0.5-5%, or 1.0-3%.

In a second aspect, the present invention relates to a method for providing a dermal and/or transdermal patch, such as a pain relief patch according to the first aspect.

In a third aspect, the present invention pertains to a dermal and/or transdermal patch provided by a method according to the second aspect.

In a fourth aspect, the present invention concerns a receptacle comprising a dermal and/or transdermal patch according to the first, third, seventh, or eighth aspect.

In a fifth aspect, the present invention relates to a kit comprising a receptacle according to the fourth aspect, and optionally, comprising an instruction for use.

In a sixth aspect, the present invention pertains to a method for pain management or treatment. comprising application of a dermal and/or transdermal patch according to the first, third, seventh, or eighth aspect.

In a seventh aspect, the present invention concerns a patch according to the first, third or eighth aspect for use as a medicament and/or therapeutic agent.

In an eighth aspect, the present invention concerns a CBD-comprising patch, such as a pain-relief patch as disclosed herein, wherein the CBD used in the formulation is crystalline. In some embodiments, the CBD is of type A (needle-like crystals) and/or capable of forming needle-like crystals.

In a ninth aspect, the present invention pertains to a dosage regimen, comprising administering a patch, such as a pain-relief patch, in particular CBD-comprising patch as disclosed herein. In some embodiments, the CBD is of “type A” and/or capable of forming needle-like crystals.

### BRIEF DESCRIPTION OF THE DRAWINGS/FIGURES

FIG. 1 depicts a visual analogue scale ranging from 0 to 10 for describing pain.

FIG. 2 is a schematic drawing of a dermal and/or transdermal patch 100, comprising a backing layer 10, usually impermeable, an adhesive layer 20 comprising active ingredients; and a release liner 30.

FIG. 3 discloses a safety data sheet for a SiO2-comprising polyethylene terephthalate (PET) film material e.g. suitable as a release liner.

FIG. 4 discloses a technical data sheet for a 110 μm polyester (PE) suitable e.g. as a backing layer.

FIGS. 5 and 6 disclose details concerning DURO-TAK 380-3954, a self-curing acrylic solution suitable for providing an adhesive layer 20 as disclosed herein.

FIG. 7 discloses a technical data sheet for a 50 g/sqm hydroentangled, non-woven, white, cross web, thermofixed PES (polyester) material suitable e.g. as a backing layer.

FIG. 8 discloses a technical data sheet for a 12 μm polyester (PE) suitable e.g. as a backing layer or release liner according to the present invention backing layer.

FIG. 9: microscope picture of cannabinol (CBD) forming needle-like crystals. The CBD crystals were sourced from www.enecta.com.

FIG. 10: microscope picture of cannabinol (CBD) forming cluster- or bunch-like crystals. The CBD crystals were sourced from www.pharma-hemp.com.

### DETAILED DESCRIPTION OF THE INVENTION

### Definitions

In the context of the present invention, the singular form of a word may include the plural, and vice versa, unless the context clearly dictates otherwise. Thus, the references “a,” “an” and “the” are generally inclusive of the plurals of the respective terms. For example, reference to “an ingredient” or “a method” may include a plurality of such “ingredients” or “methods.”

Similarly, the words “comprise,” “comprises,” and “comprising” are to be interpreted inclusively rather than exclusively. Embodiments provided by the present disclosure may lack any element that is not specifically disclosed herein. Thus, a disclosure of an embodiment defined using the term “comprising” is also a disclosure of embodiments “consisting essentially of” and “consisting of the disclosed components”. Thus, the term “comprising” is generally to be interpreted as specifying the presence of the stated parts, steps, features, or components, but does not exclude the presence of one or more additional parts, steps, features, or components. For example, a composition comprising a chemical compound may thus comprise additional chemical compounds.

Generally, compositions as disclosed herein may comprise one or more pharmaceutically acceptable carrier(s), excipient(s), stabilizer(s) or the like.

Where used herein, terms like “for example”, “e.g.” or “such as”, particularly when followed by a listing of terms, are merely exemplary and illustrative, and should not be deemed to be exclusive or comprehensive. Any embodiment disclosed herein may be combined with any other embodiment disclosed herein.

Unless expressed otherwise, all percentages expressed herein are by weight of the total weight of the composition. Thus, unless indicated otherwise, “%” indicates “% weight/weight (w/w)”, also called “weight %” or “% by weight”.

In the context of the present invention, the terms “about”, “around”, “approximately” or the symbol “˜” can be used interchangeably, and are meant to comprise variations and/or uncertainties generally accepted in the field, e.g. comprising analytical errors and the like. Thus “about” may also indicate measuring uncertainty commonly experienced in the art, which can be in the order of magnitude of e.g. +/−1, 2, 5, 10, or even 20 percent (%). Furthermore, “about” may be understood to refer to numbers in a range of numerals, for example the range of +/−20, +/−15, +/−10, +/−5, +/−2, +/−1, +/−0.5, +/−0.1% of the referenced number. Moreover, all numerical ranges herein should be understood to include all integers, whole or fractions, within the range.

As used herein, the term “in some embodiments” is meant to comprise “in one embodiment”. “in some embodiments”, and “in one or more embodiments”.

In the context of the present invention, the terms “subject” or “patient” can be used interchangeably, and are meant to comprise a human, animal and/or mammal. In particular, a human subject can e.g. be selected from one or more of: female, male, senior, adult, adolescent, child, or infant. An animal subject can e.g. be selected from pet, husbandry, mammal, reptile, bird, and/or animal in a zoo.

Generally, compositions as disclosed herein, in particular patches such as pain-relief patches may comprise one or more pharmaceutically acceptable adjuvant(s), such as pharmaceutically acceptable carrier(s), excipient(s), stabilizer(s), salt(s) and/or buffer(s) or the like.

In the context of the present invention, the term “treatment” is meant as an act aiming at alleviating, lessen, improving and/or curing any symptom(s), condition(s), or disease(s) in a subject. The effect of the treatment may also comprise reduction in pain and/or discomfort. A treatment may also result in a faster recovery and/or healing compared to a control. A further effect of a treatment may also comprise a recovery/healing with less complications compared to a control. A control can e.g. be no treatment or treatment with a placebo. Generally, a “treatment” in the present context comprises topical application of a skin patch, usually formulated as a pain relief patch, as further elucidated herein.

“Pain” can be described as a distressing feeling often caused by intense or damaging stimuli. The International Association for the Study of Pain defines pain as “an unpleasant sensory and emotional experience associated with, or resembling that associated with, actual or potential tissue damage.” In medical diagnosis, pain is regarded as a symptom of an underlying condition.

“Ache” can be used when describing a continuous feeling of pain in a part of the body, such as headache or muscle ache. The terms “ache” and “pain” can be used interchangeably herein.

Pain may motivate the subject to withdraw from damaging situations, to protect a damaged body part while it heals, and/or to avoid similar experiences in the future. Most pain resolves once the noxious stimulus is removed and the body has healed, but it may persist despite removal of the stimulus and apparent healing of the body. Pain may also arise in the absence of any detectable stimulus, damage or disease. Pain is the most common reason for physician consultation in most developed countries. It is a major symptom in many medical conditions, and can interfere with a person's quality of life and general functioning.

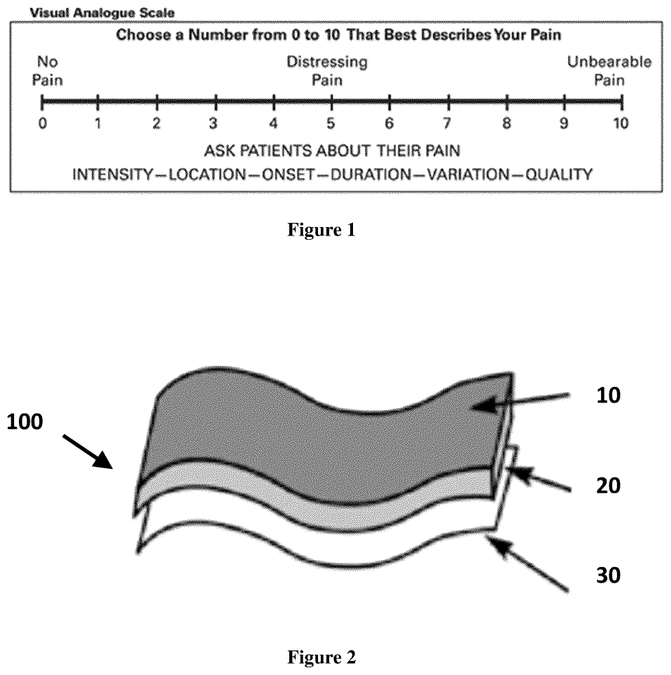

Pain can e.g. be measured according to a visual analogue scale, as presented in FIG. 1, which is regarded as a common, reproducible tool in the assessment of pain and pain relief. Alternatively, the visual analogue scale can also be a continuous line anchored by verbal descriptors, one for each extreme of pain where a higher score indicates greater pain intensity. It is usually 10 cm in length with or without intermediate descriptors as to avoid markings of scores around a preferred numeric value. When applied as a pain descriptor, these anchors are often ‘no pain’ and ‘worst imaginable pain”. Cut-offs for pain classification have been recommended as no pain (0-4 mm), mild pain (5-44 mm), moderate pain (45-74 mm) and severe pain (75-100 mm).

In the context of the present invention, the skin patches disclosed herein can be useful in the treatment and/or amelioration of pain, aches and/or conditions concerning and/or related to e.g. the following symptoms, conditions, tissues and/or body parts, such as muscle, joint, tendon, cartilage, nerve and/or skin: acute pain, inflammation, gout, bruise, sprains, lumbago, still shoulders, chronic pain, arthritis, pain in joints, such as wrist, elbow, shoulder joint, spine, lumbar, knee, ankle, finger, toe, menstrual pain, soreness and/or pain in muscles (e.g. calf muscle, thigh muscle, upper back and lower back, sheath muscle, delta muscle, three-headed arm extender, two-headed elbow, broad back muscle, serrated pectoral muscle, hamstring muscles, outer oblique abdominal muscle, flounder muscle, two-headed calf muscle, shin muscle, quadriceps muscle, large pectoral muscle) e.g. after workout, back muscle pain (e.g. due to wrong posture), backache, neck pain (wrong posture) and/or neckache.

Use of a skin patch according to the present invention may result in one or more of the following effects:
Pain reduction;
Muscle relaxation;
Cooling effect;
Reduction in need for medication, such as NSAID;
No first pass effect (through lever);
No or limited drug interaction;
Increased mobility; and/or
Increased life quality;
including any combinations thereof.

No first pass effect—also known as first-pass metabolism or presystemic metabolism is the generally undesirable phenomenon of drug metabolism, whereby the concentration of a drug, specifically when administered orally, is greatly reduced before it reaches the systemic circulation.

No (or limited) drug interaction concerns the advantage of a local treatment, wherein active ingredients do not (or only in limited amounts) enter the bloodstream and inner organs, such as lever.

A “dermal patch” is an adhesive patch, also called “skin patch” that is placed on the skin to deliver one or more active ingredients to and/or into the skin.

A “transdermal patch” is an adhesive or skin patch that is placed on the skin to deliver one or more active ingredients through the skin into the bloodstream.

A skin patch formulated to provide pain relief and/or soothing is called “pain relief patch” herein. It can be formulated as a dermal or transdermal patch.

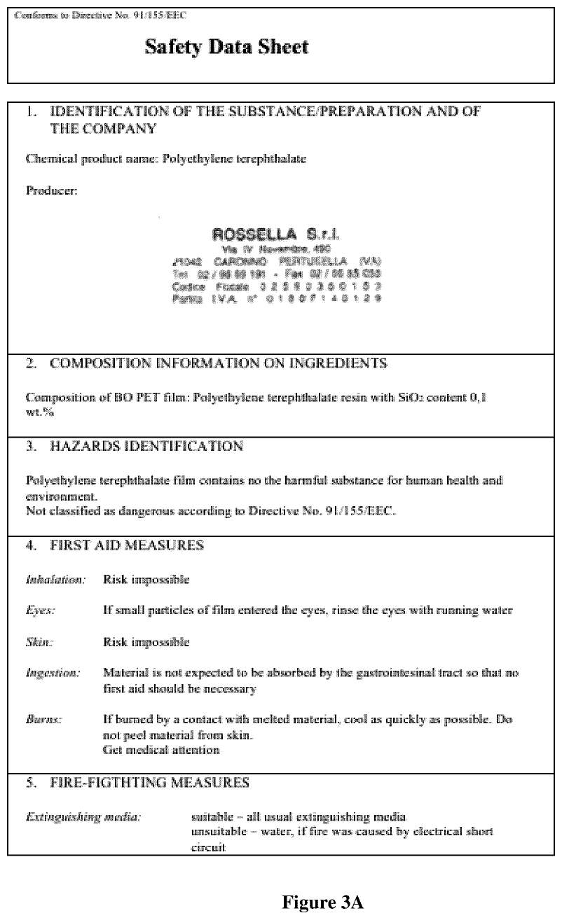

In the context of the present invention, a skin patch, such as a dermal and/or transdermal patch 100 may comprise, as e.g. illustrated in FIG. 2, a backing layer 10, an adhesive layer 20, and optionally, a release liner 30.

The backing layer 10 can be non-woven fabric support, usually flexible to allow some movement. In some embodiments, the backing layer is, or is kinesio tape, or is comparable in function to a kinesio tape commonly used for treatment of e.g. sport-related injuries. The backing layer provides protection of the areas of muscle or joint that are covered by the patch. Different strength and/or flexibilities of backing layers will provide patches with different effects, depending on the intended use (e.g. kinesio tape effect or not). Suitable materials for a backing layer may e.g. comprise cotton, stretch cotton, stretch viscose, and/or polyester.

The adhesive layer 20 comprises the active ingredients and sticks to the skin, and allows for the ingredients to get into contact with the skin upon application of the patch. The adhesive layer provides a suitable environment thereto, such as pH and moisture. The adhesive layer can be formulated in a bio-compatible manner. Usually, the adhesive layer will comprise natural extracts. The careful choice of ingredients provides release of relevant, active ingredients contained in the adhesive matrix, such as CBD and Arnica, while providing an emollient, moisturising and/or antioxidant effect on the skin. Furthermore, the formulation of the adhesive layer aims at reducing discomfort and/or risk of irritation upon removal of the plaster. On the other hand, the adhesive strength must be sufficient to ensure proper fit of the patch after application, and allow for sufficient movement of skin and/or muscles to provide a minimum of discomfort.

Furthermore, the adhesive layer is formulated with sufficient strength and/or adhesion to the backing layer that the patch can be peeled off after use by manipulating the backing layer, without leaving the adhesive layer on the skin.

The release liner 30 protects the adhesive layer from damage, such as from contamination, and/or physical damage. Suitable materials for a release liner may comprise siliconized polyethylene terephthalate (PET) or siliconized paper.

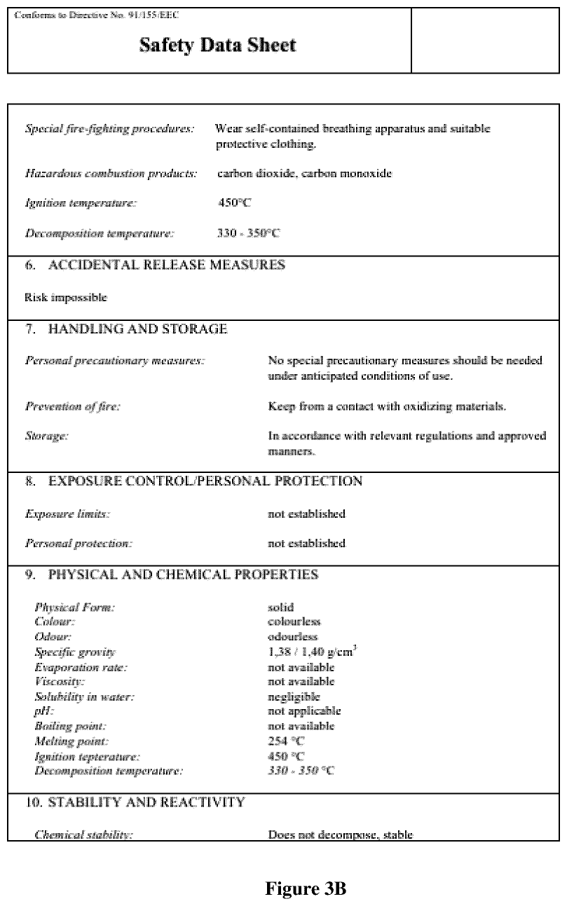

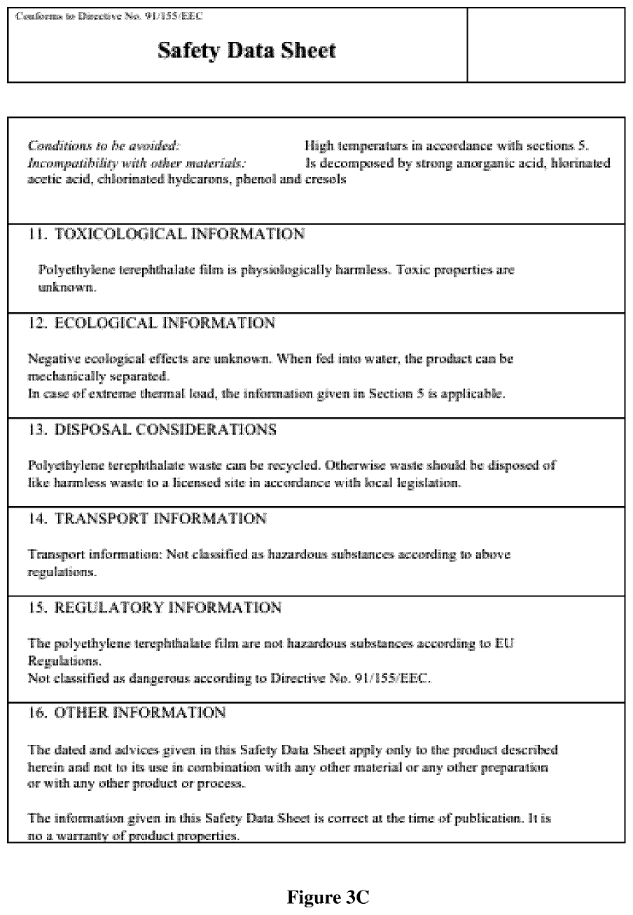

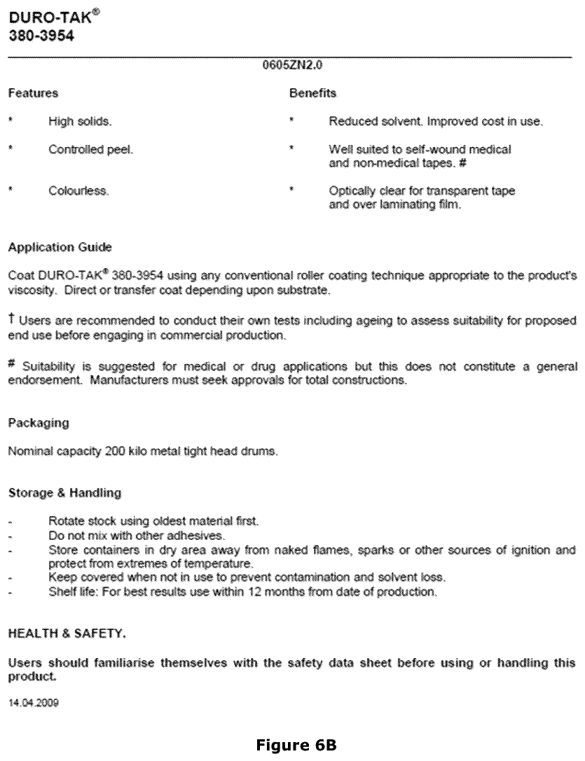

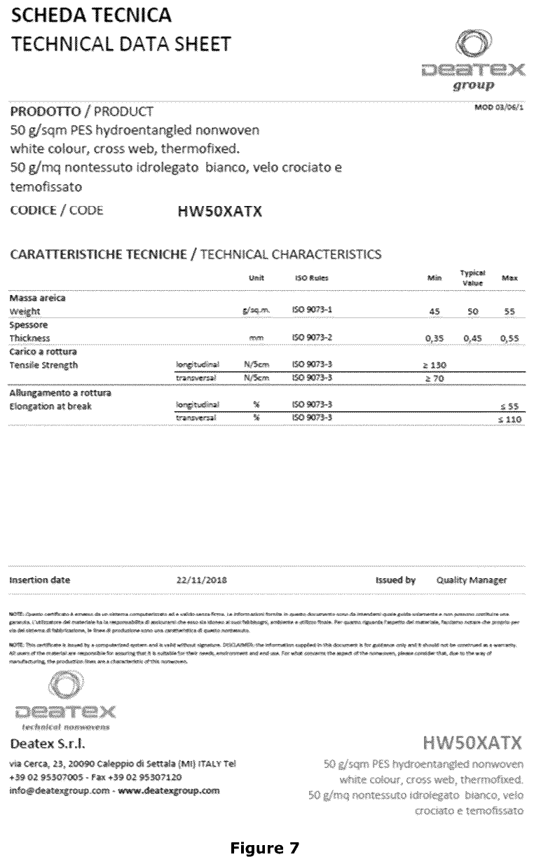

Examples for suitable materials for release liners or backing layers are e.g. disclosed in FIGS. 3, 4, 7 and 8 in greater detail.

In a first aspect, the present invention concerns a skin patch, such as a dermal and/or transdermal patch, optionally formulated as a pain relief patch, said patch comprising a backing layer, an adhesive layer, and optionally a release liner (30), said adhesive layer comprising (by weight):
Acrylates copolymer in an amount of at least 60, 70, 80, or 90%;
One or more cannabinoid(s) present in an amount of 0.1-20%, such as 0.1-10%, or 0.2-5%;
Arnica extract and/or camphor in an amount of 0.2-6%, such as 0.5-5%, or 1.0-3%;
Menthol in an amount of 0.2-6%, such as 0.5-5%, or 1.0-3%; and depending on the presence of Arnica extract or camphor in the adhesive layer, such a patch can also be called “Arnica extract patch” or “camphor patch” herein.

The Arnica extract patch is used for applications, where the presence of camphor is not desirable, such as when the cooling effect of camphor (see below) is not desirable. The camphor patch is used for applications, where the presence of Arnica extract is not desirable, such as allergy and/or sensitivity towards such plant extracts. It is believed that the presence of camphor provides a desired effect, such as by the cooling effect, which can reduce the pain perception by “moving” the sensation from pain- to temperature sensors in the skin. In some embodiments, both Arnica and camphor are present in a “combi patch”, thereby providing a combined effect. In some embodiments, this combined effect provides a surprisingly stronger effect than could be expected from a simple combination of both components.

Usually, there is no need for e.g. water and/or oil in the adhesive layer. However, in some embodiments water can be provided up to 100% of the total weight of the adhesive layer composition. In some embodiments, oil can be provided up to 100% of the total weight of the adhesive layer composition. In some embodiments, water and oil can be provided up to 100% of the total weight of the adhesive layer composition.

Generally, the presence of oil(s) and/or fat(s) is not desired in cannabinoid-comprising patches, in particular CBD, CBN, and/or CBG-comprising patches in the context of a composition formulated to be applied onto the skin of a subject. In some embodiments, the adhesive layer composition is not formulated as an oil-in-water emulsion or water-in-oil emulsion. Thus, in some embodiments, the composition comprises no, or insignificant amounts, e.g. than 1.0, 0.5 or 0.1% (w/w) oil, such as edible oil, dehydrated oil, and/or dehydrated edible oil. This may seem counter intuitive, as oils/fats are commonly used to keep the skin soft and smooth. However, in the present invention it is believed the potential downs side of a wound treatment formulation without fat(s)/oil(s) are more than outweighed by the current cannabinoid-comprising recipes. Without wanting to be bound by any theory, it is believed that the presence of such fat(s) and/or oil(s) contributes negatively with respect to the efficacy of the formulation, as CBD, CBN, and/or CBG is hydrophobic, and oil(s)/fat(s) will form a kind of barrier and/or layer on the skin, thereby impeding relevant active compounds from being able to actively participating in the healing and/or pain relieving process. Furthermore, oil(s) and/or fat(s) may even have a negative influence on the properties of the adhesive layer, such as reduction in adherence.

Consequently, in some embodiments, a CBD-comprising composition as disclosed herein comprises no or only minute amounts of oil(s) and/or fat(s). In some embodiments, the composition comprises less than 1.0, 0.5, 0.1% oil(s) and/or fat(s). In some embodiments, the composition does not comprise one or more of: (i) oil, such as edible oil, (ii) fat, such as edible fat. Generally, compositions disclosed herein do not comprise an oil-in-water or water-in-oil emulsion. In some embodiments, a skin patch does not comprise added oil(s) and/or fat(s). “Added oil(s) and/or fat(s)” is to be understood as oil(s) and/or fat(s) that are not provided by the main ingredients of the skin patch, i.e. acrylates copolymer, cannabinoid(s), arnica extract, camphor, and menthol.

In some embodiments, the adhesive layer composition is formulated such that a defined pH is provided. Generally, a neutral, near neutral, and/or slightly acidic pH, such as a pH mimicking the pH of the skin is often preferred, such as a pH of around 6.0-6.8, or around 6.5, such as 6.5±0.20, 6.25±0.25, or 6.0±0.25. In some embodiments, an adhesive layer composition can be formulated with a pH of 5-7, 5-6, 5.5-6.5, or around 6. In some embodiment, the pH is around 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In some embodiments, the pH can be around 5.2-5.8, 5.6-5.7 or around 5.5. In some embodiments, the pH is around 6-6.5. In some embodiments, the pH is 5.0 or lower. In some embodiments, the pH is 7.0 or higher.

Provision of a defined pH can be achieved using methods known in the field, comprising addition of one or more acid(s), base(s), salt(s) of said acid(s) and/or base(s), buffering agent(s) and/or pH-stabilizer(s), including any combination thereof. In some embodiments, citric acid, in particular citric acid monohydrate is used in this context. In some embodiments, other pharmaceutically acceptable acid(s) or base(s) including their salts can be used. In some embodiments, triethanolamine and/or citrate/citric acid are used in the provision and/or maintenance of the desired pH. In some embodiments, an alkanolamine such as aminomethyl propanol (AMP) is used as a buffering agent. However, in some embodiments, there is no pH adjusting agent present, such as skin patches comprising no water and/or no free water. A measurement of pH may comprise moisturizing the adhesive layer of the skin patch with distilled water.

However, as common production methods for providing skin patches comprise the application of vacuum and/or heat, usually moderate heat, to remove solvent present in the acrylates copolymers the resulting skin patch will usually not comprise any free water.

The by far dominant constituent of the adhesive layer is acrylates copolymer. “Acrylates Copolymer” is a general term for copolymers of two or more monomers consisting of acrylic acid, methacrylic acid or one of their simple esters. Other copolymers of acrylic acid and other monomers (Ammonium Acrylates Copolymer, Ammonium VA/Acrylates Copolymer, Sodium Acrylates Copolymer, Ethylene/Acrylic Acid Copolymer, Ethylene/Calcium Acrylate Copolymer, Ethylene/Magnesium Acrylate Copolymer, Ethylene/Sodium Acrylate Copolymer, Ethylene/Zinc Acrylate Copolymer, Ethylene/Acrylic Acid/VA Copolymer, Acrylates/VP Copolymer, Acrylates/VA Copolymer, Steareth-10 Allyl Ether/Acrylates Copolymer, Acrylates/Steareth-50 Acrylate Copolymer, Acrylates/Steareth-20 Methacrylate Copolymer, Acrylates/Ammonium Methacrylate Copolymer, Styrene/Acrylates Copolymer, Styrene/Acrylates/Ammonium Methacrylate Copolymer, Ammonium Styrene/Acrylates Copolymer, Sodium Styrene/Acrylates Copolymer, Acrylates/Hydroxyesters Acrylates Copolymer, Methacryloyl Ethyl Betaine/Acrylates Copolymer, Lauryl Acrylate/VA Copolymer, VA/Butyl Maleate/Isobornyl Acrylate Copolymer, Ethylene/Methacrylate Copolymer, Vinyl Caprolactam/VP/Dimethylaminoethyl Methacrylate Copolymer, Sodium Acrylates/Acrolein Copolymer, VP/Dimethylaminoethylmethacrylate Copolymer, AMP-Acrylates Copolymer) are similar to Acrylates Copolymer in their function in cosmetics and personal care products. Polymers of acrylic acid and its salts (Polyacrylic Acid, Ammonium Polyacrylate, Potassium Aluminum Polyacrylate, Potassium Polyacrylate, Sodium Polyacrylate) also have similar properties and functions and can be used instead in some embodiments. In cosmetics and personal care products, Acrylates Copolymer and the related copolymers and polymers are used in a wide variety of product types.

Acrylates copolymer is/are known in the art and can be used to provide an adhesive layer with the desired properties, such as: stability, skin-friendly and/or skin-compatible, adherence/attachment to the backing layer, compatibility with the further ingredients of e.g. camphor and/or Arnica extract batch, adherence/attachment to the skin, can be peeled-off reliably from the skin, as the adhesive layer sticks better to the backing layer than the skin).

Generally, acrylates copolymer will often be present in an amount of at least 70, 80, or 90% (by weight) of the adhesive layer. In some embodiments, it can also be less than 70% by weight, but usually at least 60%. In some embodiments, acrylates copolymer is provided “up to 100%” with respect to the combined weight of the components/ingredients/constituents of the adhesive layer.

One or more cannabinoid(s), such as CBD, CBG and/or CBN are usually present in an amount of 0.1-20%, such as 0.1-10%, or 0.2-5%.

Cannabidiol (CBD), CAS no. 3956-29-1 is a non-psychoactive cannabinoid. It can be provided in different purities, and is usually extracted from Cannabis sativa by methods known in the art. In the context of the present invention, CBD with a high degree of purity is generally preferred, such as “crystalline” CBD, comprising neither oil nor further cannabinoids, such as psychoactive or non-psychoactive cannabinoids in significant amounts.

In particular, when absence of oil(s) and/or fat(s) is desired, common sources of CBD, such as CBD-comprising oils are not desirable. Thus, in some embodiment, CBD is provided in essentially pure form, such as in crystalline or powder form and/or with a purity of 95%, 98%, 99%, 99.5%, 99.8% or more than 99.8%. Without wanting to be bound by any theory, it is believed that the use of CBD in crystalline may further contribute in a positive fashion, such as that less CBD is required to provide a similar effect compared to a crude CBD preparation. This is surprising, as according to general belief, further cannabinoids present in such crude CBD preparations are believed to provide a synergistic effect.

In some embodiments, the CBD comprises less than 1.0, 0.5, 0.2, or 0.1% of any further cannabinoid. Preferably, the CBD does not comprise any psychoactive cannabinoid, such as THC. In some embodiments, the CBD comprises less than 0.2 or 0.1 of a psychoactive cannabinoid, such as THC.

Cannabinol (CBN), CAS no. 521-35-7 is considered a mildly psychoactive cannabinoid found only in trace amounts in Cannabis. CBN can be provided from tetrahydrocannabinol (THC).

In particular, when absence of oil(s) and/or fat(s) is desired, common sources of CBN, such as CBN-comprising oils are not desirable. Thus, in some embodiments, CBN is provided in essentially pure form, such as in crystalline or powder form and/or with a purity of 95%, 98%. 99%, 99.5%, 99.8% or more than 99.8%. Without wanting to be bound by any theory, it is believed that the use of CBN in crystalline may further contribute in a positive fashion, such as that less CBN is required to provide a similar effect compared to a crude CBN preparation. This is surprising, as according to general belief, further cannabinoids present in such crude CBN preparations are believed to provide a synergistic effect.

In some embodiments, the CBN comprises less than 1.0, 0.5, 0.2, or 0.1% of any further cannabinoid. Preferably, the CBN does not comprise any, or any further psychoactive cannabinoid, such as THC. In some embodiments, the CBN comprises less than 0.2 or 0.1 of a psychoactive cannabinoid, such as THC.

Cannabigerol (CBG), CAS no. 25654-31-3 is not considered to be a psychoactive cannabinoid. CBG is a minor constituent of cannabis. During plant growth, most of the cannabigerol is converted into other cannabinoids, primarily tetrahydrocannabinol (THC) or cannabidiol (CBD), leaving about 1% cannabigerol in the plant.

In particular, when absence of oil(s) and/or fat(s) is desired, common sources of CBG, such as CBG-comprising oils are not desirable. Thus, in some embodiments, CBG is provided in essentially pure form, such as in crystalline or powder form and/or with a purity of 95%, 98%, 99%, 99.5%, 99.8% or more than 99.8%. Without wanting to be bound by any theory, it is believed that the use of CBG in crystalline may further contribute in a positive fashion, such as that less CBG is required to provide a similar effect compared to a crude CBG preparation. This is surprising, as according to general belief, further cannabinoids present in such crude CBG preparations are believed to provide a synergistic effect.

In some embodiments, the CBN comprises less than 1.0, 0.5, 0.2, or 0.1% of any further cannabinoid. Preferably, the CBN does not comprise any psychoactive cannabinoid, such as THC. In some embodiments, the CBN comprises less than 0.2 or 0.1 of a psychoactive cannabinoid, such as THC. Commonly, CBD, CBN, and/or CBG are provided in an amount of 4-12% (by weight) in skin patches according to the present invention. CBD, CBN, and/or CBG are preferably provided in pure form, such as characterized by: CBDV around or below 0.25% or 0.5%, CBDA around or below 0.25% or 0.5%, THC around or below 0.025% or 0.1%. For CBD, CBN and/or CBG are around to below 0.1% or 0.5% each. For CBN, CBD and/or CBG are around to below 0.1% or 0.5% each. For CBG, CBD and/or CBN are around to below 0.1% or 0.5% each.

CBD, CBN or CBG formulations of suitable purity are commercially available, and can e.g. be provided from www.enecta.com.

Arnica extract, CAS no. 68990-11-4, EC no. 273-579-2, is a plant extract provided from Arnica montana. Arnica extracts can e.g. be provided by CO2 extraction or other extraction methods known in the art. In some embodiments, Arnica extract is a powder. In some embodiments, Arnica extract can be fluid, such as a liquid comprising solvents and/or extractants. The US Food and Drug Administration has classified Arnica montana as an unsafe herb because of its toxicity. It should not be taken orally or applied to broken skin where absorption can occur. Arnica may irritate mucous membranes and may elicit stomach pain, diarrhea, and vomiting. Furthermore, it may produce contact dermatitis when applied to skin.

Commonly, Arnica extract is provided in an amount of 1-3% (by weight) in skin patches according to the present invention.

Camphor is a waxy, flammable, transparent solid with a strong aroma. Camphor is believed to be a parasympatholytic agent which acts as a non-competitive nicotinic antagonist at nAChRs. It is a terpenoid found in some plants, such as the camphor laurel (Cinnamomum camphora), and related trees. Also Rosemary leaves (Rosmarinus officinalis) contain 0.05 to 0.5% camphor, while camphorweed (Heterotheca) contains some 5%. A major source of camphor in Asia is camphor basil. Natural camphor can be extracted by distilling the leaves and bark of e.g. Cinnamomum camphora. Camphor can also be synthetically produced from oil of turpentine. The molecule has two possible enantiomers, the naturally occurring (+)-camphor ((1R,4R)-bornan-2-one), CAS no. 464-49-3, and (−)-camphor ((1S,4S)-bornan-2-one), CAS no. 464-48-2. The racemic (+/−) camphor has CAS no. 76-22-2. Usually, in the context of the present invention, the natural camphor is preferred, thus in some embodiments, the camphor is provided from a natural source as (+) enantiomer. However, in some embodiments, camphor can be provided synthetically, and is in the (+) conformation. In some embodiments, camphor is provided synthetically, and is in the racemic conformation. In some embodiments, camphor is provided synthetically, and is in the (−) or conformation.

In some embodiments, camphor is provided as natural camphor in solid form. In some embodiments, camphor is provided as natural camphor as camphor oil, which can be provided by steam extraction and rectification under vacuum and filter pressing. It can be coloured. White camphor oil has e.g. CAS no. 8008-51-3.

Commonly, camphor is provided in an amount of 1-3% (by weight) in skin patches according to the present invention.

Menthol is an organic compound that can be obtained from the oils of corn mint, peppermint, or other mints. It can also be synthesized. It is a waxy, crystalline substance, clear or white in color, which is solid at room temperature, but melts slightly above room temperature.

The main form of menthol occurring in nature is (−)-menthol, also termed L-menthol with assigned the (1R,2S,5R) configuration, with CAS no. CAS 2216-51-5 and EC no. 218-690-9. Menthol has local anaesthetic and counterirritant qualities, and it is widely used to relieve minor throat irritation. Menthol also acts as a weak k-opioid receptor agonist.

Menthol is believed to provide a so-called “counter-irritant effect” on the skin, especially for rheumatic complaints. This “counter-stimulus” due to the menthol, which leads to local irritation, in this case a cold stimulus, and is used in the area of application of the medical device to deflect pain from one part of the body to the point of the counter-stimulus. Since it helps refresh and revitalize, menthol can be used in numerous ways—whether it is a cooling patch for headaches or a soothing skin lotion to treat overexposure from the sun, itching and inflammation. It can be ingested, inhaled or applied topically to the skin. Topically, it is used for pain and inflammation. Menthol gives a sensation of cooling by activating TRPM8 without any actual fall in temperature in that area. This brings down the inflammation in the area. Of course that is why cooling the injury with ice works as well. Menthol provides a cooling sensation when applied to the skin, which helps relieve pain in the tissues underneath the skin. Menthol topical (for use on the skin) is used to provide temporary relief of minor arthritis pain, backache, muscles or joint pain, or painful bruises.

Commonly, menthol is provided in an amount of 1-3% (by weight) in skin patches according to the present invention.

In some embodiments, a skin patch, such as a dermal and/or transdermal patch comprising a backing layer (10), an adhesive layer (20), and optionally a release liner (30), said adhesive layer (20) comprising:
One or more cannabinoid(s) present in an amount of 0.1-20%, such as 0.1-10%, or 0.2-5% (by weight);
Arnica extract and/or camphor in an amount of 0.2-6%, such as 0.5-5%, or 1.0-3% (by weight);
Menthol in an amount of 0.2-6%, such as 0.5-5%, or 1.0-3% (by weight); and
Acrylates copolymer in an amount of at least 60, 70, 80, or 90% (by weight).

In some embodiments, an “Arnica skin patch” is provided, comprising Arnica extract, but no camphor.

In some embodiments, an “Camphor skin patch” is provided, comprising camphor, but no Arnica extract.

In some embodiments, the skin patch comprises Arnica extract and camphor, such as in a total amount of 0.2-6%, such as 0.5-5%, or 1.0-3% (by weight).

In some embodiments, the ration of Arnica extract to camphor by weight is around 1:1, such as in the range of 1.5:1 to 1:1.5. In some embodiments, the Arnica extract to camphor ratio is around 10:1-5:1; 5:1-3:1; 3:1-2:1; 2:1-1:1. In some embodiments, the camphor to Arnica extract to ratio is around 10:1-5:1; 5:1-3:1; 3:1-2:1; 2:1-1:1.

In some embodiments, the patch is, and/or is formulated a pain relief patch.

In some embodiments, the cannabinoid(s) is or comprise(s) cannabidiol (CBD).

In some embodiments, the cannabinoid(s) is or comprise(s) cannabigerol (CBG), and/or cannabinol (CBN).

In some embodiments, the cannabinoid(s) is or comprises CBD and CBG; or CBD and CBN.

In some embodiments, the amount or concentration of CBD (by weight) exceeds the amount or concentration of CBG or CBN (by weight).

In some embodiments, the ratio of CBD:CBG or CBD:CBN by weight is in the range of 10:1-5:1; 5:1-4:1; 4:1-3:1; 3:1-2:1; 2:1-1.5:1; 1.5:1-1.2:1; 1.2:1-1.1:1; or 1.1:1-1.01-1.

In some embodiments, the ratio of CBD:CBG or CBD:CBN by weight is at least 1.01:1; 1.1:1; 1.2:1; 1.3:1; 1.4:1; 1:5:1; 2:1; 3:1; 4:1; 5:1; or 10:1.

In some embodiments, the one or more cannabinoid(s) is/are provided in crystalline and/or pure form.

In some embodiments, the one or more cannabinoid(s) is/are provided dissolved in an oil, such as hemp oil.

In some embodiments, the one or more cannabinoid(s) is/are not provided dissolved in an oil, such as hemp oil.

In some embodiments, the cannabinoid(s) comprise(s) less than 1.0%, 0.5%, 0.2%, or 0.1% (by weight) of Tetrahydrocannabinol (THC), and/or any other psychoactive cannabinoid(s).

In some embodiments, a patch is provided comprising: CBD in an amount of 4-12% (by weight); Arnica extract in an amount of 1-3% (by weight); Menthol in an amount of 1-3% (by weight); and Acrylates copolymer in an amount of at least 80, or 90% (by weight).

In some embodiments, a patch is provided comprising: CBD in an amount of 4-12% (by weight); camphor in an amount of 1-3% (by weight); Menthol in an amount of 1-3% (by weight); and Acrylates copolymer in an amount of at least 80, or 90% (by weight).

In some embodiments, a patch is provided comprising: CBD in an amount of 4-12% (by weight); Arnica extract and camphor in a combined amount of 1-3% (by weight); Menthol in an amount of 1-3% (by weight); and Acrylates copolymer in an amount of at least 80, or 90% (by weight).

In some embodiments, such a patch may further comprise: CBG in an amount of 0.5-4% (by weight) and/or CBN in an amount of 0.5-4% (by weight).

In some embodiments, a patch is provided, not comprising a glycosaminoglycan and/or an acceptable salt thereof.

In some embodiments, a patch is provided, not comprising one or more C8-C22 fatty acid(s).

In some embodiments, a patch is provided, not comprising a sulfoxide.

In some embodiments, the patch comprises around 0.2-2.0, 0.3-1.0, 0.4-0.8, 0.5-0.65 or around 0.6 mg/cm2 cannabinoid(s), such as CBD, CBG, and/or CBN. In some embodiments, the patch comprises 0.57 mg cannabinoid(s)/cm2. In some embodiments, the patch comprises 0.2-2.0, 0.3-1.0, 0.4-0.8, 0.5-0.65 mg/cm2 or around 0.6 mg, such as 0.57 mg CBD. In some embodiments, the patch comprises 0.2-2.0, 0.3-1.0, 0.4-0.8, 0.5-0.65 mg/cm2 or around 0.6 mg, such as 0.57 mg CBD and CBG, such as 0.43 mg CBD and 0.14 mg CBG. In some embodiments, the patch comprises 0.2-2.0, 0.3-1.0, 0.4-0.8, 0.5-0.65 mg/cm2 or around 0.6 mg, such as 0.57 mg CBD and CBN, such as 0.43 mg CBD and 0.14 mg CBN. In some embodiments, the ratio of CBD:CBG or CBD:CBN by weight is in the range of 10:1-5:1; 5:1-4:1; 4:1-3:1; 3:1-2:1; 2:1-1.5:1; 1.5:1-1.2:1; 1.2:1-1.1:1; or 1.1:1-1.01-1.

In some embodiments, the patch comprises 0.01-1.0, 0.02-0.5, 0.05-0.25, 0.10-0.15, or around 0.14 mg Arnica extract/cm2.

In some embodiments, the patch comprises 0.01-1.0, 0.02-0.5, 0.05-0.25, 0.10-0.15, or around 0.14 mg Camphor/cm2.

In some embodiments, the patch comprises 0.01-1.0, 0.02-0.5, 0.05-0.25, 0.10-0.15, or around 0.14 mg Menthol/cm2.

In some embodiments, the patch size is around 5-10, 10-20, 20-40, or 40-100 cm2. In some embodiments, the patch size is around 5, 7.5, 10, 12.5, 15, 17.5, 20, 25, 30, 35, 40, 45, 50, 55, 60, 65, 70, 75, 80, 85, 90, 95, or 100 cm2.

In some embodiments, the adhesive layer is provided with a thickness or amount of around 30-200, 50-150, 80-100, or around 90 g adhesive layer/m2. This In some embodiments, the adhesive layer is in weight/cm2 around 1-100, 2-50, 5-25, 8-12, or around 9 or 10 mg adhesive layer/cm2.

In some embodiments the CBD used in the provision of the patch, such as the pain-relief patch is crystalline, such as “type A CBD” as disclosed herein. In some embodiments, said CBD is provided as—or capable of forming—needle-like crystals.

In some embodiments, a CBD-comprising composition according to the first aspect may comprise a further cannabinoid, such as a one or more cannabinoid(s) selected from: THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), including one or more cannabinoids of the following types: CBG-type, CBC-type, “CBD-type other than CBD”. THC-type, CBN-type, CBE-type, iso-THC-type, CBL-type, CBT-type, including any combination(s) thereof. Such further cannabinoid may comprise hallucinogenic and/or non-hallucinogenic cannabinoids. Generally, non-hallucinogenic cannabinoids are preferred in order to avoid undesired side-effects upon use or treatment with composition(s) comprising such compounds, in particular when they are present in physiologically active amounts.

Further suitable concentrations and/or concentration ranges may be disclosed herein.

Concerning the CBD used in the preparation or formulation of a CBD-comprising composition, such as the adhesive layer of a skin patch as disclosed herein, in some embodiments, the CBD used in the provision of the composition is crystalline.

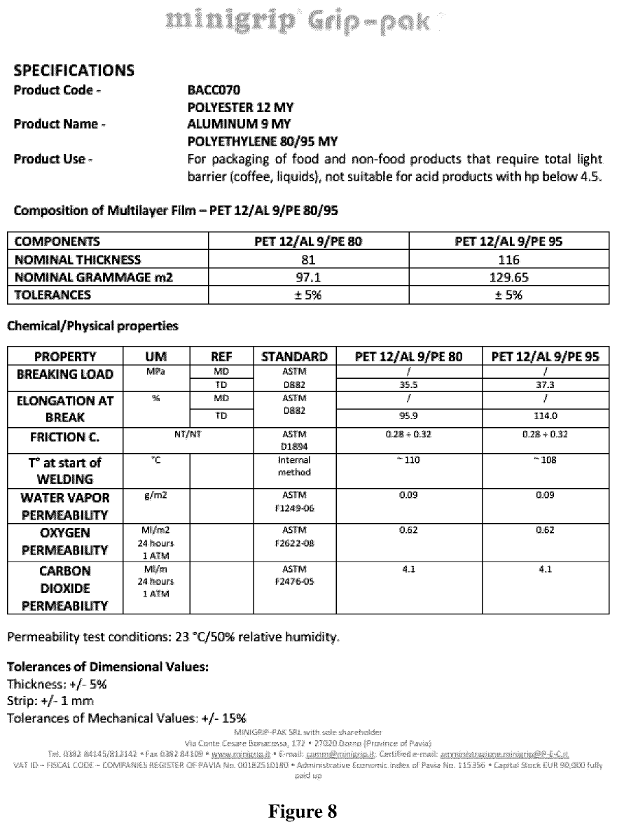

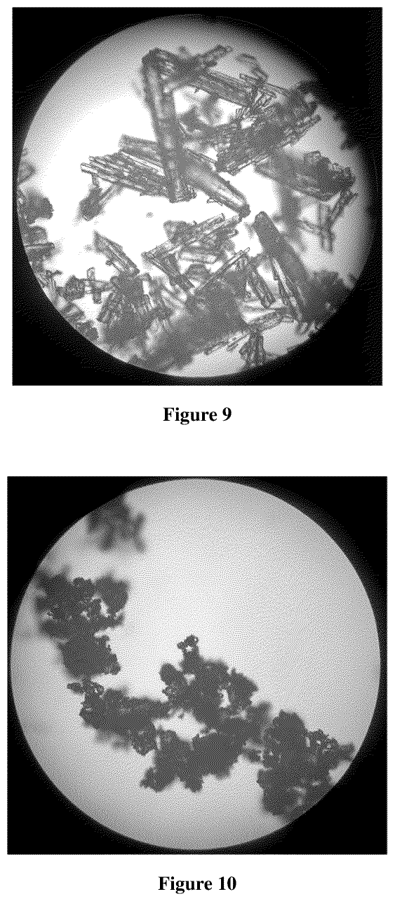

In some embodiments, the CBD used for providing a composition as disclosed above is characterized by one or more features, such as the crystal structure and/or conformation. It has been observed by the inventors, see e.g. Example 6, that CBD with a needle-like crystal structure (=crystal structure A; see FIG. 9), surprisingly and unexpectedly, appears significantly more potent than CBD with a different crystal structure, a non-needle like structure, also termed “bunch-like or “cluster-like” herein (=crystal structure B; see FIG. 10).

In some embodiments, the CBD possesses, when crystalline, or is capable of forming a needle-like crystal structure. In some embodiments, CBD of crystal structure A (or capable of forming needle-like crystals) is at least 1.2, 1.5, 2, 3, 4, 5, 7.5, 10, 15 or 20 times more “potent” or “active” on a weight/weight basis than CBD of crystal structure B (or capable of forming cluster/bunch-like crystals). Often, the “A-type” CBD is at least 2.5, 5, 7.5, or 10 times more “potent” on a weight/weight basis than “B-type” CBD.

The “potency” and/or “activity” of “type A” CBD can e.g. be determined by comparing the effect using essentially identical compositions with the exception of the type and/or quantity of CBD used. Said potency, activity and or effect can e.g. be determined essentially as disclosed in the Examples by replacing the placebo with the “B-type” CBD comprising formulation. Alternatively, the potency can be determined in the quantity of CBD needed to provide the same effect, such as pain relief. In some embodiments, the use of a more potent CBD results in an increase in pain relief and/or reduction in pain. In some embodiments, the use of a more potent CBD allows for a reduction of the quantity of CBD used in the formulation of skin patch to ensure a comparable effect in pain relief. In some embodiments, the use of a more potent CBD allows for a reduction of the quantity of formulation necessary to provide a comparable effect, such as a pain relief and/or pain reduction.

CBD of crystal structure A, or CBD capable of forming needle-like crystals, is also called “type A CBD” herein, while CBD of crystal structure B, or CBD capable of forming “bunch-like or “cluster-like” crystals is called “type B CBD”. In some embodiments, the CBD is “type A CBD”. Often, “type A CBD” is preferred in contrast to “type B CBD”.

It can be speculated, if the CBD needs to be in an active form, such one or more specific conformation(s) in order to be active upon administration to a subject, such as in a topical formulation, e.g a skin patch as disclosed herein. Lack of activity or potency can also be caused by a lower uptake rate and/or difficulties in passing through the skin.

Without wanting to be bound by any theory, it is believed that the difference in crystal structure may be caused by a different molecular structure, such as a different conformation. This could e.g. be due to a failure of the subject's body to recognize the “wrong” CBD conformation or the like. It is conceivable that the differences in CBD crystal structure are caused by a different extraction process. In particular, the CBD disclosed in FIG. 1 was provided by an extraction process, comprising extraction with isopropanol, distillation and crystallization with heptane, while the CBD disclosed in FIG. 2 was provided by critical CO2 extraction.

Generally, crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:
Extracting hemp or cannabis with e.g. isopropanol to produce an extract rich in cannabinoids, THC, CBD and terpenes
Evaporating the solvent portion of the extract to generate a substantially solvent-free extract
Distilling the substantially solvent-free extract to isolate the CBD, and
Crystallizing the distilled, isolated CBD to produce a crystallized, isolated CBD and one or more recrystallization(s) if needed by the use of a suitable organic solvent, such as an alkane, e.g. heptane, commonly followed by
Solvent removal by e.g. vacuum drying. to remove volatile remnants.

Thus, in some embodiments, the CBD crystals used in the formulation of the topical composition, such as the skin patch composition, e.g. pain-relief patch are needle-like crystals, such as crystals shown in FIG. 9. Likewise, in some embodiments, the CBD crystals used in the formulation of the topical composition are not cluster- or bunch-shaped, such as crystals similar to crystals shown in FIG. 10.

In some embodiments, the CBD crystals used in the formulation of the topical composition are not provided by an extraction method comprising critical CO2 extraction.

In some embodiments, the CBD crystals used in the formulation of the topical composition are provided by a method comprising extraction with a C3-C4 alcohol, such as isopropanol, and one or more crystallisations steps with a C6-C8 alkane, such as heptane. In some embodiments, the C3-C4 alcohol is isopropanol. In some embodiments, the C6-C8 alkane is heptane. In some embodiments, the C3-C4 alcohol is isopropanol, and the C6-C8 alkane is heptane. This combination is believed to provide CBD crystals of satisfactory quality, such as absence or reduction in inhibitors and/or the desired conformation of the CBD.

In some embodiments, a suitable CBD product can be obtained when the CBD crystals are provided by a method comprising critical CO2 extraction and one or more crystallisations steps with a C6-C8 alkane, such as heptane.

As seen in Table 1, it can be seen that the Cannabinoid profile of type A and type B CBD can be rather similar.

TABLE 1
Analysis of CBD of crystal structure A versus crystal structure B

| Cannabinoid profile | Type A | Type B |
|---|---|---|
| CBD | 99.33% | 98.60% |
| CBDV | 0.39% | 0.19% |
| CBDA | 0.01% | n.d. |
| CBG | n.d. | n.d. |
| CBN | 0.04% | n.d. |
| THC | n.d. | n.d. |

n.d. not detected;
type A CBD was sourced from Enecta,
type B CBD was sourced from Pharma Hemp

It is, however, also conceivable that the differences in crystal structure, can relate to and be caused by different extraction processes. Different crystal structures can also be indicative of different concentrations of “CBD inhibitors”, and/or different concentrations of “CBD enhancers”. In some embodiments, terpenes, such as naturally occurring terpenes, in particular terpenes found in plants, such as in Cannabis sativa, act as CBD inhibitors, which is not desirable.

Thus, in some embodiments, CBD of crystal structure B alias “type B CBD” can be converted to CBD of crystal structure A alias “type A CBD” (and/or CBD capable of forming crystal structure A) by an organic extraction step and/or recrystallisation step. In such embodiments, it is conceivable that the change in crystal structure is related to the presence of inhibitors that are reduced significantly in the additional extraction and/or crystallization step(s). Alternatively, the organic extraction step may provide a change in conformation of the CBD, rendering it more active again. In some embodiments, recrystallization with heptane can change the B-type CBD into A-type CBD.

In some embodiments, CBD of crystal structure B has been provided by critical CO2 extraction, such as CBD crystals provided by www.pharma-hemp.com and/or following a similar extraction protocol as said manufacturer.

In some embodiments, presence of terpenes and/or terpenoids, in particular Cannabis sativa terpenes or in a CBD-comprising topical composition as disclosed herein, provides one or more undesirable effect(s), such as one or more of: reduced efficiency or potency, inability or reduced ability to recognize the CBD, need for a higher CBD formulation for obtaining similar effect, increase in non-CBD cannabinoids in the formulation. In some embodiments, said composition comprises 0.0001% or less, 0.001% or less, 0.01% or less, or 0.1% or less terpenes, in particular Cannabis sativa terpenes, by weight.

In some embodiments, the crystalline CBD does not comprise significant amounts of terpenes, such as less than 0.1, less than 0.05, less than 0.02, less than 0.01, less than 0.005, less than 0.002, less than 0.001% terpenes by weight.

It is also conceivable that other plant components, such as terpenoids can act as inhibitors. In some embodiments the presence of terpenoids, such as Cannabis sativa terpenoids can be undesirable. In some embodiments, the crystalline CBD does not comprise significant amounts of terpenoids, such as less than 0.1, less than 0.05, less than 0.02, less than 0.01, less than 0.005, less than 0.002, less than 0.001% terpenoids by weight.

In some embodiments, the use of CBD having or capable of providing crystals of crystal structure A, such as shown in FIG. 9 in a CBD-comprising composition as disclosed herein, provides a positive effect, such as one or more of: increased efficiency, possibility to reduce total amount of CBD in the formulation, the subject needs less topical composition, such as skin patch formulation to achieve the same effect, improved recognition and/or CBD uptake by the subject's body, reduction in non-CBD cannabinoids in the formulation and/or other impurities.

Generally, compositions according to the first aspect can be provided using methods, procedures and/or unit operations known in the art. In some embodiments, compositions according to the first aspect can be provided as shown herein, such as in the second aspect.

In a second aspect, the present invention relates to a method for providing a skin patch, such as dermal and/or transdermal patch, such as a pain relief patch according to the first aspect.

Such a method may comprise the acts or steps of:
Providing CBD, CBG and/or CBN;
Providing Arnica extract and/or Camphor;
Providing Menthol;
Combining and mixing components (a), (b) and (c) to provide an active ingredient mixture;
Providing Acrylates copolymer comprising solvents;
Combining and mixing the active ingredient mixture from step (d) with the Acrylates copolymer from step (e) to provide a crude adhesive mixture;
Depositing a layer of the crude adhesive mixture from step (f) on a backing layer or release liner material;
Removing solvents from the deposited crude adhesive mixture by moderate heat (such as 40-120° C., or 40-100° C., or 40-80° C., e.g. a temperature higher than room temperature and preferably lower than 80, 70, 60, 50 or 45° C.) and/or vacuum.

In some embodiments, said method further comprises the act of punching the backing layer to provide individual patches.

In some embodiments, said method further comprises the act of depositing a release liner on the exposed surface of the adhesive layer.

In some embodiments, said method further comprises the act of providing a protective enclosure, such as by packaging.

The active ingredients (i) cannabinoid(s), (ii) Arnica extract and/or camphor, and (iii) menthol are weighed and mixed to provide an active ingredient mixture.

The cannabinoid or cannabinoids (CBD, CBN, and/or CBN) are mixed with the Arnica extract and/or camphor.

In some embodiments, the CBD is crystalline CBD. In some embodiments, the CBD is “type A CBD”. Often, the use of “type A CBD” is preferred in contrast to “type B CBD” or other types of CBD.

An appropriate amount of Acrylates copolymer is provided, such as a mixture of Acrylates copolymer comprising organic solvents.

Acrylates copolymer comprising solvents—such as FMD 2027 provided from Fastmed Italia with following characteristics: solid contents (60 min 150° C.); solvents ˜48% ethyl acetate, ˜26% EtOH, ˜25% heptane and ˜0.5% Pentanedione—are weighed and combined with the active ingredient mixture to provide the crude adhesive layer mixture.

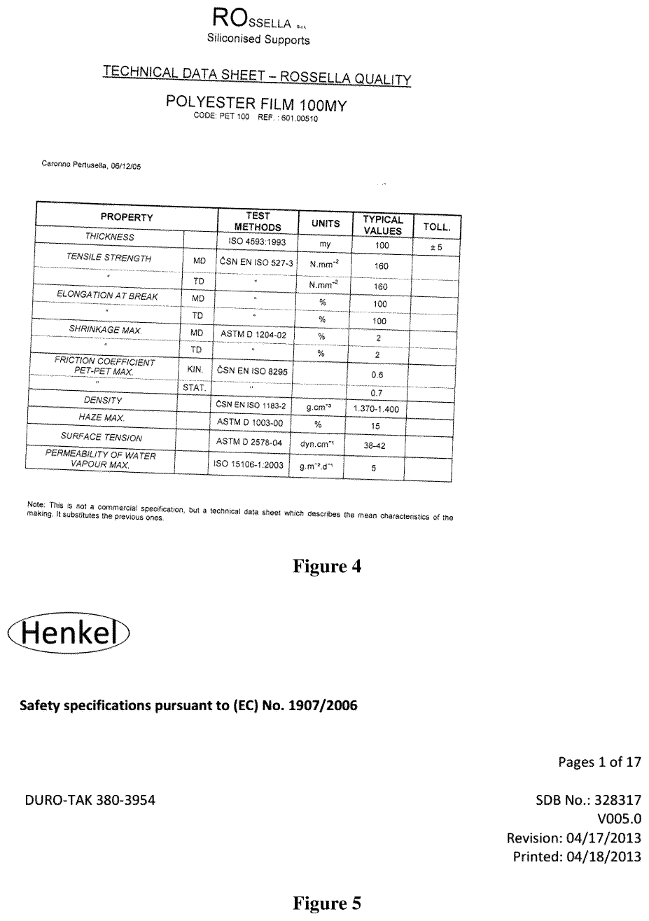

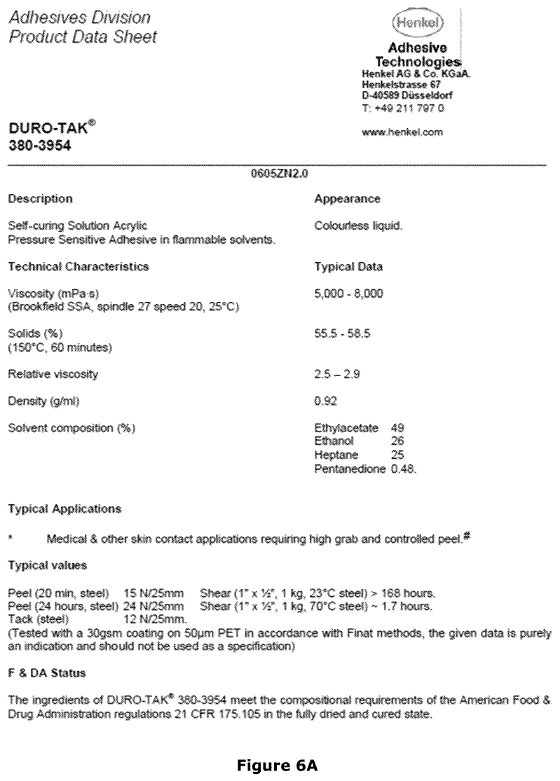

In some embodiments, the adhesive layer is provided using DURO-TAK, such as DURO-TAK 380-3954 (see e.g. FIGS. 5 and 6 for details), a self-curing acrylate composition.

In some embodiments, the active ingredient mixture is added to the Acrylates copolymer mixture tank and mixed for a period of 15 minutes through a vertical mixer in the tank.

In some embodiments, the crude matrix comprising of Acrylates copolymer and active ingredients is collected by means of a pump and deposited on the release liner, the reel of which is mounted on the machine. The thickness of the coating is achieved by an adjustable doctor blade. The coated release liner sheet then passes through the ovens and is laminated at the end of the machine with the backing layer sheet and rewound onto a reel (parent reel). The adhesive matrix consisting of Acrylates Copolymer and Active ingredients will then stick to the backing layer sheet, due to the fact that the release liner sheet is siliconized.

Generally, when the release liner is removed to use the patch, the adhesive matrix will remain stuck to the backing layer.

The crude adhesive mixture is deposited on the release liner (alternatively, the backing layer by the use of equipment customary in the field. Usually, this process comprises means for removing solvents present in the crude adhesive layer mixture, predominantly deriving from the solvent in the Acrylates copolymer.

A common production method for providing skin patches comprising the application of vacuum and/or heat, usually moderate heat such as 40-120° C., or 40-100° C., or 40-80° C. preferably below 80, 70, 60 to 50° C. to remove solvent present in the acrylates copolymers. As evaporating solvents may provide a cooling, the person skilled in the art can adjust the process parameters to provide conditions that not only protect the active ingredients, but also the backing layer and/or release liner from damage and/or deterioration. Such a critical temperature can e.g. be 50 or 60° C., depending on the material used.

Suitable patch sized can e.g. be 5×7 cm, but they can also be larger or smaller, as also disclosed herein. Often, the area of the backing layer will be slightly larger than the area of the adhesive layer.

Upon removal of the solvents, such as by the application of moderate heat for a sufficient amount of time, the skin patch is provided. Such a skin patch can e.g. be deposited with 90 g/m2. To protect the adhesive layer of the skin patch from damage, contamination and/or other undesirable environmental influences, a release liner can be placed on the exposed surface of the adhesive layer, or vice versa, the backing layer, depending on the method used.

In some embodiments, the parent reel is then mechanically cut into several smaller (smaller width) reels depending on the size of the final patch release liner. Finally, the small reels are mounted on the die-cutting machine that forms the patches.

In some embodiments, release liner and/or backing layer are siliconized.

In some embodiments, release liner and/or backing layer comprise a material, as disclosed in one or more of FIG. 3, 4, 7, or 8.

In some embodiments, packaging is performed manually or automatically, such as in sealed PET/alu/PE bags. In some embodiments, packaging is be performed using methods, materials and devices known in the field.

In a third aspect, the present invention pertains to a dermal and/or transdermal patch provided by a method according to the second aspect.

In a fourth aspect, the present invention concerns a receptacle comprising a skin patch, such as a dermal and/or transdermal patch according to the first, third or seventh aspect.

In some embodiments, the skin patch is provided in an enclosure, such as a sealed PET/alu/PE bag or pouch. The patches can be sealed individually, or in groups of e.g. 2, 4 or more patches. In some embodiments, the receptacle is such a sealed bag or pouch. In some embodiment, said receptacle comprises one or more bags/pouches.

In some embodiments, the receptacle according the receptacle provides a protective enclosure.

In some embodiments, the receptacle comprises more than 1 dermal patches, such as 2, 4, 5, 7 or 10 patches.

In a fifth aspect, the present invention relates to a kit comprising one or more patch(es) according to any one of the first, third or seventh aspect, and/or one or more receptacles according to the fourth aspect, and optionally, an instruction for use.

In some embodiments, such a kit will comprise an instruction for use.

In some embodiments, a kit may comprise a multitude of patches, such as 2, 4, 5, 7 or 10 patches. In some embodiments, such a kit may comprise more than 10 patches.

In some embodiments, said patches are provided with individual protective enclosures/receptacle(s).

In some embodiments, said enclosure(s) protect(s) said patch(es) from one or more of: ambient air, oxidation, decomposition, physical damage, light, UV, or contamination, including any combination thereof.

In a sixth aspect, the present invention pertains to a method for pain management or treatment of pain comprising application of a skin patch, such as a dermal and/or transdermal patch according to the first, third or seventh aspect.

In some embodiments, said pain and/or discomfort is related to one or more of muscle, joint, tendon, cartilage, nerve and/or skin.

In some embodiments, skin patches as disclosed herein can be used in the treatment and/or amelioration of pain, aches and/or conditions concerning and/or related to e.g. acute pain, inflammation, gout, bruise, sprains, lumbago, still shoulders, chronic pain, arthritis, pain in joints, such as wrist, elbow, shoulder joint, spine, lumbar, knee, ankle, finger, toe, menstrual pain, soreness and/or pain in muscles (e.g. calf muscle, thigh muscle, upper back and lower back, sheath muscle, delta muscle, three-headed arm extender, two-headed elbow, broad back muscle, serrated pectoral muscle, hamstring muscles, outer oblique abdominal muscle, flounder muscle, two-headed calf muscle, shin muscle, quadriceps muscle, large pectoral muscle) e.g. after workout, back muscle pain (e.g. due to wrong posture), backache, neck pain (wrong posture) and/or neckache.

In some embodiments, the pain, condition and/or discomfort is selected from: acute pain, inflammation, gout, bruise, sprains, lumbago, still shoulders, chronic pain, arthritis, pain in joints, such as wrist, elbow, shoulder joint, spine, lumbar, knee, ankle, finger, toe, menstrual pain, soreness and/or pain in muscles (e.g. calf muscle, thigh muscle, upper back and lower back, sheath muscle, delta muscle, three-headed arm extender, two-headed elbow, broad back muscle, serrated pectoral muscle, hamstring muscles, outer oblique abdominal muscle, flounder muscle, two-headed calf muscle, shin muscle, quadriceps muscle, large pectoral muscle) e.g. after workout, back muscle pain (e.g. due to wrong posture), backache, neck pain (wrong posture), and neckache.

In some embodiments, 1, 2, or 3 patches are applied per 24 h per affected area.

In some embodiments, the patch comprises around 0.2-2.0, 0.3-1.0, 0.4-0.8, 0.5-0.65 mg/cm² or around 0.6 or 0.57 mg CBD, CBG, and/or CBN/cm², such as:
0.2-2.0, 0.3-1.0, 0.4-0.8, 0.5-0.65 mg/cm² or around 0.6 mg/cm², such as 0.57 mg/cm² CBD;
0.2-2.0, 0.3-1.0, 0.4-0.8, 0.5-0.65 mg/cm² or around 0.6 mg/cm², such as 0.57 mg/cm² CBD and CBG, such as 0.43 mg/cm² CBD and 0.14 mg/cm² CBG; and/or
0.2-2.0, 0.3-1.0, 0.4-0.8, 0.5-0.65 mg/cm² or around 0.6 mg/cm², such as 0.57 mg/cm² CBD and CBN, such as 0.43 mg/cm² CBD and 0.14 mg/cm² CBN.

In some embodiments, the ratio of CBD:CBG or CBD:CBN by weight is in the range of 10:1-5:1; 5:1-4:1; 4:1-3:1; 3:1-2:1; 2:1-1.5:1; 1.5:1-1.2:1; 1.2:1-1.1:1; or 1.1:1-1.01-1.

In some embodiments, the patch comprises around 0.01-1.0, 0.02-0.5, 0.05-0.25, 0.10-0.15, or around 0.14 mg Arnica extract/cm2.

In some embodiments, the patch comprises around 0.01-1.0, 0.02-0.5, 0.05-0.25, 0.10-0.15, or around 0.14 mg Camphor/cm2.

In some embodiments, the patch comprises around 0.01-1.0, 0.02-0.5, 0.05-0.25, 0.1.0-0.15, or around 0.14 mg Menthol/cm2.

In some embodiments, the patch size is around 5-10, 10-20, 20-40, or 40-100 cm2.

In some embodiments, the adhesive layer is provided with 30-200, 50-150, 80-100, or around 90 g adhesive layer/m2.

In a seventh aspect, the present invention concerns a patch according to the first, third or eighth aspect for use as a medicament and/or therapeutic agent, such as in the treatment of pain, pain management and/or discomfort

In some embodiments, said pain and/or discomfort is related to one or more of muscle, joint, tendon, cartilage, nerve and/or skin.

In some embodiments, skin patches as disclosed herein can be used in the treatment and/or amelioration of pain, aches and/or conditions concerning and/or related to e.g. acute pain, inflammation, gout, bruise, sprains, lumbago, still shoulders, chronic pain, arthritis, pain in joints, such as wrist, elbow, shoulder joint, spine, lumbar, knee, ankle, finger, toe, menstrual pain, soreness and/or pain in muscles (e.g. calf muscle, thigh muscle, upper back and lower back, sheath muscle, delta muscle, three-headed arm extender, two-headed elbow, broad back muscle, serrated pectoral muscle, hamstring muscles, outer oblique abdominal muscle, flounder muscle, two-headed calf muscle, shin muscle, quadriceps muscle, large pectoral muscle) e.g. after workout, back muscle pain (e.g. due to wrong posture), backache, neck pain (wrong posture) and/or neckache.

In some embodiments, 1, 2, or 3 patches are applied per 24 h per affected area.

In some embodiments, the patch comprises CBD, CBG, and/or CBN in a concentration of around 0.2-2.0, 0.3-1.0, 0.4-0.8, 0.5-0.65 mg/cm², or around 0.6 mg/cm², such as 0.57 mg CBD, CBG, and/or CBN/cm². In some embodiments, the patch comprises e.g.:
0.2-2.0, 0.3-1.0, 0.4-0.8, 0.5-0.65 mg/cm² or around 0.6 mg/cm², such as 0.57 mg/cm² CBD;
0.2-2.0, 0.3-1.0, 0.4-0.8, 0.5-0.65 mg/cm² or around 0.6 mg/cm², such as 0.57 mg/cm² CBD and CBG, such as 0.43 mg/cm² CBD and 0.14 mg/cm² CBG per cm²; and/or
0.2-2.0, 0.3-1.0, 0.4-0.8, 0.5-0.65 mg/cm² or around 0.6 mg/cm², such as 0.57 mg/cm² CBD and CBN, such as 0.43 mg/cm² CBD and 0.14 mg/cm²CBN.

In some embodiments, the ratio of CBD:CBG or CBD:CBN by weight is in the range of 10:1-5:1; 5:1-4:1; 4:1-3:1; 3:1-2:1; 2:1-1.5:1; 1.5:1-1.2:1; 1.2:1-1.1:1; or 1.1:1-1.01-1. In some embodiments, the patch comprises around 0.01-1.0, 0.02-0.5, 0.05-0.25, 0.10-0.15, or around 0.14 mg Arnica extract/cm2.

In some embodiments, the patch comprises around 0.01-1.0, 0.02-0.5, 0.05-0.25, 0.10-0.15, or around 0.14 mg Camphor/cm2.

In some embodiments, the patch comprises around 0.01-1.0, 0.02-0.5, 0.05-0.25, 0.10-0.15, or around 0.14 mg Menthol/cm2.

In some embodiments, the patch size is around 5-10, 10-20, 20-40, or 40-100 cm2.

In some embodiments, the adhesive layer is provided with 30-200, 50-150, 80-100, or around 90 g adhesive layer/m2.

In some embodiments, a skin patch according to the present invention provides one or more of the following effects:
Pain reduction;
Muscle relaxation;
Cooling effect;
Reduction in need for medication, such as NSAID;
No first pass effect (through lever);
No or limited drug interaction;
Increased mobility; and/or
Increased life quality;
including any combinations thereof.

In an eighth aspect, the present invention concerns a CBD-comprising composition, such as a patch according to any one of the preceding aspects, wherein the CBD used in the formulation is crystalline and/or of “type A”. In some embodiments, said composition is a topical composition as disclosed herein, such as skin patch formulated for pain/discomfort relief. e.g. according to the first or third aspect. In some embodiments, the CBD is of type A (needle-like crystals) or capable of forming needle-like crystals as disclosed herein, e.g. in the first aspect and/or in the Examples.

In a ninth aspect, the present invention pertains to a dosage regimen, comprising administering a topical composition, in particular CBD, CBN and/or CBG-comprising topical composition such as a skin patch disclosed herein. In some embodiments, the CBD is of “type A”.

The current invention is further exemplified in the following section. These examples are, however, not to be construed as limiting to the present invention.

### EXAMPLES

Generally, percentages are % by weight. Crystalline CBD is sourced from Enecta, unless indicated otherwise.

Cannabinoid—comprising compositions, such as CBD-comprising compositions according to the present invention can be at least in part be provided using methods, unit operations, protocols and/or know-how customary in the field. This can e.g. be performed as disclosed herein, such as according to the third aspect of the invention, and/or in particular, according to the following Examples.

### Example 1—Provision of a Skin Patch

Skin patches are provided as disclosed in the second aspect of the invention following a method comprising the steps of:
Providing CBD, CBG and/or CBN;
Providing Arnica extract and/or Camphor;
Providing Menthol;
Combining and mixing components (a), (b) and (c) to provide an active ingredient mixture;
Providing Acrylates copolymer comprising solvents;
Combining and mixing the active ingredient mixture from step (d) with the Acrylates copolymer from step (e) to provide a crude adhesive mixture;
Depositing a layer of the crude adhesive mixture from step (f) on a release liner material;
Removing solvents from the deposited crude adhesive mixture by moderate heat (such as 40-120° C., or 40-100° C., or 40-80° C., e.g. a temperature higher than room temperature and preferably lower than 80, 70, 60, 50 or 45° C.) and/or vacuum;
punching the backing layer to provide individual patches;
depositing a backing layer material on the exposed surface of the adhesive layer; and
providing a protective enclosure, such as by packaging.

Several skin patches comprising different adhesive layer compositions are provided, comprising various amounts of CBD, CBN, CBG, Camphor and/or Arnica extract (“Arnica”) as disclosed below, wherein the concentrations are approximate concentrations per 35 cm² patch, measuring approximately 5×7 cm.
A. 15 mg CBD+5 mg CBN+5 mg Arnica+5 mg Menthol
B. 15 mg CBD+5 mg CBN+5 mg Camphor+5 mg Menthol
C. 15 mg CBD+5 mg CBG+5 mg Arnica+5 mg Menthol
D. 15 mg CBD+5 mg CBG+5 mg Camphor+5 mg Menthol
E. 20 mg CBD+5 mg Arnica+5 mg Menthol

General recipe, not taking account for the amount of solvent (usually 40-60%) in the acrylate copolymer composition:

| | | | | % active | |
|---|---|---|---|---|---|
| | | | | in raw | |
| Ingredients: | % | mg | | material | Active (%) |
| Acrylates copolymer | 90.4222 or | 283.22 or | | | 90.422, |
| | 92.1322 or | 288.57 or | | | 92.1322 or |
| | 100 | 313.21 | | | 100 |
| CBD | 4.448 or | 15 or 20 | mg | 98 | 4.36 or. |
| | 6.158 | | | | 6.04 |
| CBN, CBG, or none | 0 or 1.71 | 0 or 5 | mg | 98 | 0 or. 1.68 |
| Arnica extract or | 1.71 | 5 | mg | 98 or 96 | 1.68 or |
| Camphor | | | | | 1.64 |
| Menthol | 1.71 | 5 | mg | 98 | 1.68 |

F. 20 mg CBD+5 mg Menthol
G. Placebo (only Acrylate copolymer)

In the above compositions, 15 or 20 mg CBD relates to around 4.36% or 6.04 pure CBD in the adhesive layer, respectively. For the remaining cannabinoids, 5 mg CBG or CBN corresponds to around 1.68% pure CBG or CBN in the adhesive layer. Finally, 5 mg Arnica extract or Menthol correspond to 1.68, or 1.64% pure extract or product. Acrylate polymer is added “to 100%”, such as around 90.4, 92.1 or 100%. All percentages are by weight.

The purity of the different components/ingredients may be slightly higher or lower as indicated above.

### Example 2—Application/Use of Skin Patch

### Instruction of Use:

Open the sachet and remove one of the patches and remove the release liner. Stick the adhesive side of the patch to the area to be treated, until it is well adhered. Leave it on for at least 8 hours, up to a maximum of 24 hours depending on the need, and then replace it with a new one.

The patch is single use. The patches are disposable. For external use only. Apply only to intact, clean and dry skin. Keep out of reach of children. Discontinue treatment in case of irritation or clear signs of hypersensitivity to the product. The product's functionality and safety are guaranteed by the bag's integrity.

### Example 3—Inclusion & Exclusion Criteria for Pain Study

### Inclusion Criteria:

Subjects of either sex.

Age 18-65 (both inclusive).

Subjects having at least one or more sign and symptoms of pain in joints (knee, ankle, elbow or shoulder) or in muscles (work out, menstrual pain, back, neck, shoulder, upper arm or lower arm, calf muscle, thigh muscle).

Pain score of at least 4 cm on a 10 cm linear visual analogue scale (see FIG. 1). Are free of any systemic or dermatologic disorder, which, in the opinion of the investigator, will interfere with the study results or increase the risk of adverse events.

Are willing to avoid prolonged exposure of the treatment area to ultraviolet radiation (natural or artificial) for the duration of the study.

Are willing to refrain from using any lotions, gel, balm, moisturizer, cleansers, cosmetics or creams, on the treatment areas during the treatment period.

If female of childbearing potential, must be willing to practice an acceptable form of birth control for the duration of the study.

Are willing to avoid participation in any other interventional clinical trial for the duration of this study.

### Exclusion Criteria:

Are pregnant, breast-feeding, or planning to become pregnant during the study. Patients with Rheumatoid Arthritis, Systemic Lupus Erythematosus, Psoriatic Arthritis, Gouty Arthritis.

Complications of osteoarthritis (OA), like pseudogout, apontaneous osteonecrosis of the knee, ruptured Baker cyst, bursitis, anserine bursitis (knee) interfere with the disease and treatment.

Severe Stomach infection.

Severely traumatized and/or very severe or mucosal inflammation.

Peritonsillar abscess.

Long term use (≥3 times per week within the last month or regular intake within the last 3 months before randomisation) of antiinflammatory drugs—Any long-acting or slow release analgesic intake including NonSteroidal Anti-Inflammatory Drugs (NSAIDs) within 24 hours before randomisation (e.g. piroxicam or naproxen).

Any anti-inflammatory drugs intake by systemic route within 12 hours before randomisation Any paracetamol intake within 6 hours before randomisation.

Any cold medication (decongestant, antihistamine, expectorant, antitussive) within 6 hours before randomisation.

Heavy smokers (>10 cigarettes/day).

Have open sores or open lesions in the treatment area(s).

Have any condition that, in the opinion of the investigator, would confound the safety and/or efficacy assessments of plaque psoriasis.

Have participated in any interventional clinical trial in the previous 30 days.

Have a known sensitivity to any of the constituents of the test product.

Have used, are using, or are planning to use immunosuppressive or immunomodulatory medication (i.e., biologics), including oral or parenteral corticosteroids.

Have a history of alcohol or illegal drug/substance abuse, or suspected alcohol or illegal drug/substance abuse in the past 2 years.

### Example 4—Test Results

TABLE A
Pain Patch(15 mg CBD + 5 mg CBN + 5 mg Arnica + 5 mg Menthol)

| Test person | 1 | 2 | 3 | 4 | 5 | 6 | 7 |
|---|---|---|---|---|---|---|---|
| Body place muscle or Joint with | Calf | Thigh | Knee | Knee | Neck | Upper | Knee |
| pain and if after work out (WO) | muscle | muscle | | | | back | |
| or injury (IN) or neither | | | | | | | |
| NSAID use: | T: 0 | T: 0 | T: 3 | T: 3 | T: 0 | T: 0 | T: 3 |
| 400 mg*T/day | | | | | | | |
| Test period(days) minimum | 4 | 4 | 8 | 7 | 4 | 5 | 9 |
| 4 days-max 30 days | | | | | | | |
| Pain scaled on a VAS 0-10 scale | B: 6 | B: 7 | B: 9 | B: 7 | B: 9 | B: 5 | B: 8 |
| before and after test period | A: 4 | A: 3 | A: 4 | A: 3 | A: 5 | A: 2 | A: 5 |
| Pain free exercise in min. | B: 0 | B: 0 | B: 0 | B: 0 | B: 0 | B: 0 | B: 0 |
| before and after test period | A: 15 | A: 25 | A: 10 | A: 10 | A: 0 | A: 15 | A: 10 |
| NSAID use reduced during | NA | NA | No - | Yes - | NA | NA | No - |
| test period? | | | T: 3 | T: 2 | | | T: 3 |

400 mg*T/day = 400 mg Ibuprofen*times/day

TABLE B
Pain Patch (15 mg CBD + 5 mg CBN + 5 mg Camphor + 5 mg Menthol)

| Test person | 1 | 2 | 3 | 4 | 5 | 6 | 7 |
|---|---|---|---|---|---|---|---|
| Body place muscle or Joint with | Knee | Neck | Knee | Lower | Shoulder | Ankle | Knee |
| pain and if after work out (WO) | | | | Back | | | |
| or injury (IN) or neither | | | | | | | |
| NSAID use: | T: 3 | T: 0 | T: 3 | T: 4 | T: 0 | T: 3 | T: 3 |
| 400 mg*T/day | | | | | | | |
| Test period(days) minimum | 7 | 9 | 5 | 6 | 9 | 4 | 8 |
| 4 days-max 30 days | | | | | | | |
| Pain scaled on a VAS 0-10 scale | B: 8 | B: 7 | B: 9 | B: 7 | B: 9 | B: 5 | B: 8 |
| before and after test period | A: 6 | A: 4 | A: 6 | A: 4 | A: 6 | A: 3 | A: 5 |
| Pain free exercise in min. | B: 0 | B: 0 | B: 0 | B: 0 | B: 0 | B: 5 | B: 0 |
| before and after test period | A: 15 | A: 20 | A: 20 | A: 20 | A: 15 | A: 20 | A: 15 |
| NSAID use reduced during | Yes - | NA | NO - | Yes - | NA | NO | Yes - |
| test period? | T: 2 | | T: 3 | T: 3 | | | T: 2 |

400 mg*T/day = 400 mg Ibuprofen*times/day

TABLE C
Pain Patch (15 mg CBD + 5 mg CBG + 5 mg Arnica + 5 mg Menthol)

| Test person | 1 | 2 | 3 | 4 | 5 | 6 | 7 |
|---|---|---|---|---|---|---|---|
| Body place muscle or Joint with | Thigh | Lower | Knee | Wrist | Knee | Shoulder | Knee |
| pain and if after work out (WO) | muscle | back | | | | | |
| or injury (IN) or neither | | | | | | | |
| NSAID use: | T: 0 | T: 3 | T: 4 | T: 0 | T: 4 | T: 3 | T: 3 |
| 400 mg*T/day | | | | | | | |
| Test period(days) minimum | 5 | 8 | 7 | 4 | 9 | 5 | 8 |
| 4 days-max 30 days | | | | | | | |
| Pain scaled on a VAS 0-10 scale | B: 8 | B: 7 | B: 9 | B: 7 | B: 9 | B: 5 | B: 8 |
| before and after test period | A: 3 | A: 2 | A: 3 | A: 1 | A: 4 | A: 1 | A: 3 |
| Pain free exercise in min. | B: 0 | B: 0 | B: 0 | B: 0 | B: 0 | B: 0 | B: 0 |
| before and after test period | A: 30 | A: 90 | A: 35 | A: 20 | A: 20 | A: 120 | A: 25 |
| NSAID use reduced during | NA | Yes | Yes | NA | Yes | Yes | Yes |
| test period? | | after 1 | after 1 | | after 1 | after 1 | after 1 |
| | | patch | patch | | patch | patch | patch |

400 mg*T/day = 400 mg Ibuprofen*times/day

TABLE D
Pain Patch (15 mg CBD + 5 mg CBG + 5 mg Camphor + 5 mg Menthol)

| Test person | 1 | 2 | 3 | 4 | 5 | 6 | 7 |
|---|---|---|---|---|---|---|---|
| Body place muscle or Joint with | Knee | Knee | Knee | Shoulder | Lower | Thigh | Knee |
| pain and if after work out (WO) | | | | | back | muscle | |
| or injury (IN) or neither | | | | | | | |
| NSAID use: | T: 3 | T: 3 | T: 4 | T: 3 | T: 4 | T: 0 | T: 3 |
| 400 mg*T/day | | | | | | | |
| Test period(days) minimum | 10 | 7 | 9 | 6 | 4 | 4 | 8 |
| 4 days-max 30 days | | | | | | | |
| Pain scaled on a VAS 0-10 scale | B: 9 | B: 8 | B: 7 | B: 6 | B: 7 | B: 6 | B: 7 |
| before and after test period | A: 3 | A: 2 | A: 1 | A: 0 | A: 1 | A: 0 | A: 3 |
| Pain free exercise in min. | B: 0 | B: 0 | B: 0 | B: 0 | B: 10 | B: 0 | B: 0 |
| before and after test period | A: 60 | A: 45 | A: 90 | A: 120 | A: 90 | A: 120 | A: 35 |
| NSAID use reduced during | Yes | Yes | Yes | Yes | Yes | NA | Yes |
| test period? | after 1 | after 1 | after 1 | after 1 | after 1 | | after 1 |
| | patch | patch | patch | patch | patch | | patch |

400 mg*T/day = 400 mg Ibuprofen*times/day

TABLE E
Pain Patch (20 mg CBD + 5 mg Arnica + 5 mg Menthol)

| Test person | 1 | 2 | 3 | 4 | 5 | 6 | 7 |
|---|---|---|---|---|---|---|---|
| Body place muscle or Joint with | Knee | Elbow | Knees | Knee | Wrists | Knee | Knee |
| pain and if after work out (WO) | | | | | | | |
| or injury (IN) or neither | | | | | | | |
| NSAID use: | T: 3 | T: 0 | T: 3 | T: 3 | T: 3 | T: 3 | T: 3 |
| 400 mg*T/day | | | | | | | |
| Test period(days) minimum | 9 | 6 | 8 | 7 | 8 | 8 | 7 |
| 4 days-max 30 days | | | | | | | |
| Pain scaled on a VAS 0-10 scale | B: 8 | B: 7 | B: 9 | B: 7 | B: 9 | B: 5 | B: 8 |
| before and after test period | A: 1 | A: 0 | A: 2 | A: 0 | A: 3 | A: 0 | A: 2 |
| Pain free exercise in min. | B: 0 | B: 0 | B: 0 | B: 0 | B: 0 | B: 0 | B: 0 |
| before and after test period | A: 60 | A: 120 | A: 35 | A: 120 | A: 35 | A: 120 | A: 45 |
| NSAID use reduced during | Yes | NA | Yes | Yes | Yes | Yes | Yes |
| test period? | after 1 | | after 1 | after 1 | after 1 | after 1 | after 1 |
| | patch | | patch | patch | patch | patch | patch |

400 mg*T/day = 400 mg Ibuprofen*times/day

TABLE F
Pain Patch (20 mg CBD + 5 mg Menthol)

| Test person | 1 | 2 | 3 | 4 | 5 | 6 | 7 |
|---|---|---|---|---|---|---|---|
| Body place muscle or Joint with | Lower | Shoulder | Knees | Calf | Thigh | Knee | Knee |
| pain and if after work out (WO) | Back | | | muscle | muscle | | |
| or injury (IN) or neither | | | | | | | |
| NSAID use: | T: 3 | T: 0 | T: 4 | T: 0 | T: 0 | T: 4 | T: 3 |
| 400 mg*T/day | | | | | | | |
| Test period(days) minimum | 6 | 5 | 7 | 4 | 4 | 9 | 8 |
| 4 days-max 30 days | | | | | | | |
| Pain scaled on a VAS 0-10 scale | B: 6 | B: 5 | B: 9 | B: 7 | B: 5 | B: 7 | B: 8 |
| before and after test period | A: 4 | A: 3 | A: 5 | A: 4 | A: 3 | A: 4 | A: 5 |
| Pain free exercise in min. | B: 10 | B: 0 | B: 0 | B: 0 | B: 10 | B: 0 | B: 0 |
| before and after test period | A: 25 | A: 35 | A: 25 | A: 20 | A: 45 | A: 35 | A: 25 |
| NSAID use reduced during | Yes | NA | Yes | NA | NA | Yes | Yes |
| test period? | after 1 | | after 1 | | | after 1 | after 1 |
| | patch | | patch | | | patch | patch |

400 mg*T/day = 400 mg Ibuprofen*times/day

TABLE G
Pain Patch (Placebo)

| Test person | 1 | 2 | 3 | 4 | 5 | 6 | 7 |
|---|---|---|---|---|---|---|---|
| Body place muscle or Joint with | Neck | Knee | Knee | Knee | Lower | Elbow | Thigh |
| pain and if after work out (WO) | | | | | back | | muscle |
| or injury (IN) or neither | | | | | | | |
| NSAID use: | T: 0 | T: 3 | T: 4 | T: 3 | T: 0 | T: 3 | T: 0 |
| 400 mg*T/day | | | | | | | |
| Test period(days) minimum | 4 | 4 | 4 | 4 | 4 | 4 | 4 |
| 4 days-max 30 days | | | | | | | |
| Pain scaled on a VAS 0-10 scale | B: 7 | B: 8 | B: 9 | B: 7 | B: 6 | B: 7 | B: 8 |
| before and after test period | A: 6 | A: 8 | A: 8 | A: 7 | A: 5 | A: 7 | A: 8 |
| Pain free exercise in min. | B: 10 | B: 0 | B: 0 | B: 0 | B: 10 | B: 0 | B: 0 |
| before and after test period | A: 15 | A: 0 | A: 10 | A: 0 | A: 15 | A: 0 | A: 0 |
| NSAID use reduced during | NA | No - | No - | No - | NA | No - | NA |
| test period? | | T: 3 | T: 4 | T: 3 | | T: 3 | |

400 mg*T/day = 400 mg Ibuprofen*times/day

### Example 5—Provision of CBD by Alcohol Extraction, Distillation and Crystallization

Crystalline CBD can be provided by methods and techniques known in the art, such as by methods disclosed in U.S. Ser. No. 10/413,845 and/or U.S. Ser. No. 10/414,709.

In short, crystalline CBD can be provided from hemp or cannabis (Cannabis sativa) by a method consisting essentially of:
Extracting hemp or cannabis with a solvent selected from the group consisting of propanol, isopropanol, butanol, pentanol, hexanol, heptanol, and octanol to produce an extract consisting essentially of an extracted hemp or cannabis consisting essentially of tetrahydrocannabinol, a terpene, or cannabidiol;
Evaporating the solvent portion of the extract to generate a substantially solvent-free extract comprising CBD;
Distilling the substantially solvent-free extract to isolate the CBD, and
Crystallizing the distilled, isolated CBD to produce a crystallized, isolated CBD.

Often, the crystallized, isolated CBD is subjected to vacuum drying to remove volatile remnants, in particular the solvent used in crystallizing or re-crystallizing, if needed.

In particular, a method comprising extraction with isopropanol and crystallization by the use of heptane, including one or more optional re-crystallization steps, followed by vacuum drying can provide CBD with crystal structure A, i.e. needle like crystals. Furthermore, such a CBD can be very low in undesired compounds, such as terpenes.

GC chromatography or other analytical methods known in the art can be used to monitor the process such as to ensure a high yield and/or a high purity of the desired product.

Concerning the raw material, hemp comprising e.g. 2-3% CBD is dried and ground before extraction with isopropanol, such as food grade isopropanol.

Guidance for choosing the appropriate reaction based on the boiling points or ranges of the different compounds can e.g. be found here:
www.nwsci.com/customer/docs/SKUDocs/RMR/Technical %20Data_Extractions_03.28.18.p df.

CBD with crystal structure A can e.g. be provided from www.enecta.com, and/or following a similar extraction and/or purification protocol as said manufacturer.

### Example 6—Comparison of Compositions Formulated with Different Crystalline CBDs

Two different sets of skin patches are provided according to Example 1, e.g. formulation A or F, the only difference being that the crystalline CBD used in the formulation is either of type A (needle-like crystals; FIG. 9) or type B (bunch/cluster-like; FIG. 10).

Type A crystalline CBD is sourced from Enecta, while type B CBD is sourced from Pharma Hemp.

When testing both compositions, surprisingly and unexpectedly, it is seen and/or it can be concluded that a skin patch provided with “type A” CBD is significantly more active than a comparable skin patch with “type B” CBD. Such tests can e.g. be performed, mutatis mutandis, according to Examples 1-4.

1-67. (canceled)

68. A dermal or transdermal skin patch formulated as a pain relief patch comprising a backing layer (10), and an adhesive layer (20), said adhesive layer (20) comprising:
one or more cannabinoid(s) present in an amount of 0.1-20%, 0.1-10%, or 0.2-5% (by weight), said cannabinoid(s) being selected from one or more of: cannabidiol (CBD), cannabigerol (CBG), or cannabinol (CBN), including any combination(s) thereof;
Arnica extract, camphor, or Arnica extract and camphor in an amount of 0.2-6%, 0.5-5%, or 1.0-3% (by weight);
Menthol in an amount of 0.2-6%, 0.5-5%, or 1.0-3% (by weight); and
Acrylates copolymer in an amount of at least 60, 70, 80, or 90% (by weight).

69. The skin patch according to claim 68 comprising CBD and CBG or CBD and CBN, wherein the amount of CBD exceeds the amount of CBG or CBN by weight; and wherein the ratio of CBD:CBG or CBD:CBN is in the range of 10:1-5:1; 5:1-4:1; 4:1-3:1; 3:1-2:1; 2:1-1.5:1; 1.5:1-1.2:1; 1.2:1-1.1:1; or 1.1:1-1.01-1 by weight; or at least 1.01:1; 1.1:1; 1.2:1; 1.3:1; 1.4:1; 1:5:1; 2:1; 3:1; 4:1; 5:1; or 10:1 by weight.

70. The skin patch according to claim 68, wherein the one or more cannabinoid(s) is provided in crystalline form or dissolved in an oil.

71. The skin patch according to claim 68 comprising CBD, wherein the CBD used in the skin patch is crystalline, and the CBD crystals comprise:
needle-like crystals;
CBD crystals not provided by critical CO2 extraction;
CBD crystals provided by extraction with a C3-C4 alcohol, and one or more crystallisations steps with a C6-C8 alcohol; or
any combination(s) thereof.

72. The skin patch according to claim 68, wherein the cannabinoid(s) comprise(s) less than 1.0%, 0.5%, 0.2%, or 0.1% (by weight) of a psychoactive cannabinoid, wherein said psychoactive cannabinoid comprises Tetrahydrocannabinol (THC) or other psychoactive cannabinoid.

73. The skin patch according to claim 68, comprising:
CBD in an amount of 4-12% (by weight);
Arnica extract, camphor or Arnica extract and camphor in an amount of 1-3% (by weight);
Menthol in an amount of 1-3% (by weight); and
Acrylates copolymer in an amount of at least 80% or 90% (by weight).

74. The skin patch according to claim 68, further comprising:
CBG, CBN or CBG and CBN in an amount of 0.5%-4% (by weight).

75. The skin patch according to claim 68, not comprising one or more of:
a glycosaminoglycan or an acceptable salt thereof;
one or more C8-C22 fatty acid(s);
a sulfoxide; or
any combination(s) thereof.

76. The skin patch according to claim 68, wherein the patch comprises around 0.2-2.0, 0.3-1.0, 0.4-0.8, 0.5-0.65 mg/cm² or around 0.6 mg of CBD, CBG, CBN, a combination of CBD and CBG, or a combination of CBD and CBN per cm² of the patch.

77. The skin patch according to claim 75, wherein the patch comprises around:
a. 0.43 mg/cm² CBD and 0.14 mg/cm² CBG; or
b. 0.43 mg/cm² CBD and 0.14 mg/cm² CBN.

78. The skin patch according to claim 77, wherein the patch comprises one or more of:
0.01-1.0, 0.02-0.5, 0.05-0.25, 0.10-0.15, or around 0.14 mg Arnica extract/cm²;
0.01-1.0, 0.02-0.5, 0.05-0.25, 0.10-0.15, or around 0.14 mg Camphor/cm²; or
0.01-1.0, 0.02-0.5, 0.05-0.25, 0.10-0.15, or around 0.14 mg Menthol/cm².

79. The skin patch according to claim 68, wherein the patch size is around 5-10, 10-20, 20-40, or 40-100 cm².

80. The skin patch according to claim 68, wherein the adhesive layer is in a weight/cm² of 1-100 mg, 2-50 mg, 5-25 mg, 8-12 mg, or around 9 or 10 mg of the adhesive layer/cm².

81. The skin patch according to claim 68, wherein the backing layer is, or is suitable for functioning as a tape having the characteristics of Kinesio™ tape.

82. A method for providing a skin patch comprising a backing layer and an adhesive patch according to claim 68, comprising the steps of:
a. preparing a component (a) comprising a combination of one or more cannabinoid(s) selected from CBD, CBG, or CBN;
b. preparing a component (b) comprising a combination of Arnica extract, Camphor, or Arnica extract and Camphor;
c. preparing a component (c) comprising menthol;
d. combining and mixing components (a), (b) and (c) to provide an active ingredient mixture;
e. preparing an Acrylates copolymer comprising solvents;
f. combining and mixing the active ingredient mixture from step (d) with the Acrylates copolymer of step (e) to provide a crude adhesive mixture;
g. depositing a layer of the crude adhesive mixture from step (f) on a backing layer or release liner material;
h. removing solvents from the deposited crude adhesive mixture by moderate heat or vacuum.

83. The method according to claim 82, further comprising one or more act(s) or step(s) selected from:
i. Punching the backing layer to provide individual patches;
j. Depositing a release liner or backing layer on the exposed surface of the adhesive layer; or
k. Providing a protective enclosure, such as by packaging;
including any combination(s) thereof.

84. A patch provided by a method according to claim 83.

85. A method for treating or reducing pain or discomfort to a skin area of a subject in need of pain management comprising:
applying the skin patch according to claim 68 to the skin area of the subject; and
providing treatment or reducing pain at the skin area of the subject,
wherein pain at the skin area of the subject at which the skin patch is provided is less than pain at the skin area of the subject not receiving the skin patch.

86. The method according to claim 85, wherein the pain or discomfort is related to one or more of:
muscle, joint, tendon, cartilage, nerve or skin:
pain, aches or conditions concerning or related to one or more of: acute pain, inflammation, gout, bruise, sprains, lumbago, still shoulders, chronic pain, arthritis;
pain in joints, such as wrist, elbow, shoulder joint, spine, lumbar, knee, ankle, finger, toe;
menstrual pain;
soreness or pain in muscles (e.g., calf muscle, thigh muscle, upper back and lower back, sheath muscle, delta muscle, three-headed arm extender, two-headed elbow, broad back muscle, serrated pectoral muscle, hamstring muscles, outer-oblique abdominal muscle, flounder muscle, two-headed calf muscle, shin muscle, quadriceps muscle, large pectoral muscle) e.g., after workout, back muscle pain (e.g., due to wrong posture);
backache, neck pain (wrong posture) and/or neckache; or
any combination thereof.

87. The method according to claim 85, wherein the patch size is around 5-10, 10-20, 20-40, or 40-100 cm2, or around 5, 7.5, 10, 12.5, 15, 17.5, 20, 25, 30, 35, 40, 45, 50, 55, 60, 65, 70, 75, 80, 85, 90, 95, or 100 cm2.
